(19) United States
(12) Patent Application Publication
(10) Pub. No.: US 2024/0081988 A1
(43) Pub. Date: Mar. 14, 2024

(54) IMPLANTABLE FIBERS, YARNS AND TEXTILES

(71) Applicant: EDWARDS LIFESCIENCES CORPORATION, Irvine, CA (US)

(72) Inventors: Shrojalkumar M. Desai, Tustin, CA (US); Travis Zenyo Oba, Yorba Linda, CA (US); Sam Shafigh, Irvine, CA (US); Mark Chau, Laguna Hills, CA (US); Jean-Pierre Michel Rabbah, Tustin, CA (US); Paul Kaye, Modiin (IL); Rupesh Gajanan Nawalakhe, Trabuco Canyon, CA (US); Radhika Vaid, Irvine, CA (US); Sandip Vasant Pawar, Irvine, CA (US); Gillian Irene Armstrong, Huntington Beach, CA (US); Ekaterina Tkatchouk, Huntington Beach, CA (US)

(21) Appl. No.: 18/511,999

(22) Filed: Nov. 16, 2023

Related U.S. Application Data
(63) Continuation of application No. PCT/US2022/029215, filed on May 13, 2022.
(60) Provisional application No. 63/190,198, filed on May 18, 2021.

(51) Int. Cl. A61F 2/24; A61L 17/10; A61L 27/18; C08L 75/04; D01F 1/10; D01F 6/90; D01F 6/92; D01F 6/94; D02G 1/16; D02G 3/32; D02G 3/36; D02G 3/44; D03D 1/00; D03D 15/283; D04B 1/16; D04B 1/18; D04B 21/16; D04B 21/18; D04H 1/4326; D04H 1/56; D04H 1/728

(52) U.S. Cl. CPC A61F 2/2418; A61L 17/10; A61L 27/18; C08L 75/04; D01F 1/10; D01F 6/90; D01F 6/92; D01F 6/94; D02G 1/16; D02G 3/32; D02G 3/36; D02G 3/448; D03D 1/00; D03D 15/283; D04B 1/16; D04B 1/18; D04B 21/16; D04B 21/18; D04H 1/4326; D04H 1/56; D04H 1/728; A61F 2002/075; C08L 2201/06; D10B 2331/00; D10B 2331/02; D10B 2331/04; D10B 2331/10; D10B 2509/04

(57) ABSTRACT

An implant includes a frame, and a textile mounted on the frame. The textile includes fibers formed from a mixture of a polymer and an additive molecule. The additive molecule includes a polyurethane backbone and one or more fluorinated end-groups at at least one end of the backbone. Other embodiments are also described.

### CROSS-REFERENCES TO RELATED APPLICATIONS

The present application is a Continuation of International Patent Application PCT/US2022/029215 to Desai et al., filed May 13, 2022, and titled “Implantable fibers, yarns and textiles,” which published as WO 2022/245657, and which claims priority to U.S. Provisional Patent Application 63/190,198 to Desai et al., filed May 18, 2021, and titled “Fibers, yarns, textiles, and/or implants including effector molecules.”

Each of the above applications is incorporated herein by reference in its entirety for all purposes.

### BACKGROUND

Over time, implanted medical devices may suffer from fouling and bacterial adherence and growth and/or may promote thrombogenesis. These phenomena may reduce the effectiveness of the device, and/or may cause negative outcomes for the subject in whom the device is implanted.

### SUMMARY

Some applications of the present disclosure relate to inclusion of effector molecules with (e.g., in, on, around, etc.) fibers, yarns, textiles, implants, and/or other medical devices, e.g., in order to modify their interaction with the body of a subject, such as to improve their suitability for use in medical treatments and devices (e.g., implants, etc.). For some applications, the modification involves including a fluorinated molecule (e.g., polymer) in and/or on a fiber, yarn, fabric, and/or textile.

For some applications, a molecule having a polymeric backbone and fluorinated end-groups (or end caps) is mixed, as an additive (which can be at a low concentration), with the base/structural polymer from which synthetic fibers will be formed.

For some applications, a molecule having a polymeric backbone and fluorinated end-groups is coated onto pre-formed fibers, yarns, and/or fabrics, e.g., by applying a solution and/or melt that comprises the molecule.

For some applications, a fluorinated polyphosphazene is coated onto pre-formed fibers, yarns, and/or fabrics, e.g., by applying a solution and/or melt that comprises the fluorinated polyphosphazene.

For some applications, the fluorinated molecule is coated onto one or more metallic components of a medical implant or medical device.

Some applications of the present disclosure relate to loosely woven or knit fabrics which are stabilized to be suitable for use in implants or other medical devices, and to methods for creating such fabrics.

There is provided, in accordance with some applications, a method, including forming, into a fiber, a mixture that includes: a polymer and an additive molecule that includes one or more fluorinated end-groups. The method also includes incorporating the fiber in a yarn, fabric, textile, implant, and/or medical device.

The term “yam” as used herein encompasses a variety of elongate components like yarn, sutures, thread, string, etc.

In some applications, the additive molecule includes both (i) a polymeric backbone, and (ii) fluorinated end-groups at at least one end of the backbone; and

In some applications, the polymeric backbone includes polyurethane, and forming the mixture into the fiber includes forming, into the fiber, the mixture that includes the additive molecule that includes the polymeric backbone that includes polyurethane.

In some applications, the method further includes forming the mixture.

In some applications, the polymer is a thermoplastic polymer, such as thermoplastic polyurethane, and forming the mixture into the fiber includes forming, into the fiber, the mixture that includes the thermoplastic polymer.

In some applications, the polymer is nylon, and forming the mixture into the fiber includes forming, into the fiber, the mixture that includes nylon.

In some applications, the polymer is an elastomer, and forming the mixture into the fiber includes forming, into the fiber, the mixture that includes the elastomer.

In some applications, forming the mixture into the fiber includes extruding the mixture into the fiber.

In some applications, forming the mixture into the fiber includes spinning (e.g., electrospinning, rotary jet spinning, etc.) the mixture into or with the fiber.

In some applications, incorporating the fiber in the yarn, fabric, textile, implant, and/or medical device includes incorporating the fiber in a nonwoven fabric or textile.

In some applications, incorporating the fiber in the yarn, fabric, textile, implant, and/or medical device includes incorporating the fiber in a blended fabric or textile.

In some applications, the mixture includes the additive molecule at 1-5 percent by mass, and forming the mixture into the fiber includes forming, into the fiber, the mixture that includes the additive molecule at 1-5 percent by mass.

In some applications, the mixture includes the additive molecule at 1-4 percent by mass, and forming the mixture into the fiber includes forming, into the fiber, the mixture that includes the additive molecule at 1-4 percent by mass.

In some applications, the mixture includes the additive molecule at 1-3 percent by mass, and forming the mixture into the fiber includes forming, into the fiber, the mixture that includes the additive molecule at 1-3 percent by mass.

In some applications, the mixture includes the additive molecule at 1-2 percent by mass, and forming the mixture into the fiber includes forming, into the fiber, the mixture that includes the additive molecule at 1-2 percent by mass.

In some applications, the mixture includes the additive molecule at 2-3 percent by mass, and forming the mixture into the fiber includes forming, into the fiber, the mixture that includes the additive molecule at 2-3 percent by mass.

In some applications, the mixture includes the additive molecule at 2-5 percent by mass, and forming the mixture into the fiber includes forming, into the fiber, the mixture that includes the additive molecule at 2-5 percent by mass.

In some applications, the mixture includes the additive molecule at 2-4 percent by mass, and forming the mixture into the fiber includes forming, into the fiber, the mixture that includes the additive molecule at 2-4 percent by mass.

In some applications, the mixture includes the additive molecule at 3-4 percent by mass, and forming the mixture into the fiber includes forming, into the fiber, the mixture that includes the additive molecule at 3-4 percent by mass.

In some applications, the mixture includes the additive molecule at 3-5 percent by mass, and forming the mixture into the fiber includes forming, into the fiber, the mixture that includes the additive molecule at 3-5 percent by mass.

In some applications, the mixture includes the additive molecule at 4-5 percent by mass, and forming the mixture into the fiber includes forming, into the fiber, the mixture that includes the additive molecule at 4-5 percent by mass.

In some applications, the method includes incorporating the fiber into a fabric or textile and then incorporating the fabric/textile into a medical implant or device.

In some applications, incorporating the fabric/textile into the medical implant or device includes stitching the fabric/textile to a frame of the medical implant or device.

In some applications, incorporating the fabric/textile into the medical implant or device includes forming the fabric/textile into a sleeve of the implant or device.

In some applications, the polymer is a polyester, and forming the mixture into the fiber includes forming, into the fiber, the mixture that includes the polyester.

In some applications, the polyester is polyethylene terephthalate, and forming the mixture into the fiber includes forming, into the fiber, the mixture that includes polyethylene terephthalate.

In some applications, the polyethylene terephthalate is a polyethylene terephthalate homopolymer, and forming the mixture into the fiber includes forming, into the fiber, the mixture that includes polyethylene terephthalate homopolymer.

In some applications, the polyethylene terephthalate is a polyethylene terephthalate copolymer, and forming the mixture into the fiber includes forming, into the fiber, the mixture that includes polyethylene terephthalate copolymer.

In some applications, incorporating the fiber in the yarn, fabric, textile, implant, and/or medical device includes forming a yarn that includes the fiber, and then incorporating the yarn in a fabric, textile, implant, and/or medical device.

In some applications, the yarn consists substantially of only the fiber, and forming the yarn includes forming the yarn from substantially only the fiber.

In some applications, the fiber is a first fiber, the yarn is a blend of the first fiber and a second fiber, and forming the yarn includes forming the yarn from the blend of the first fiber and the second fiber.

In some applications, forming the yarn includes spinning the yarn.

In some applications, the method further includes cutting the fiber into staple fiber, and spinning the yarn includes spinning the staple fiber into the yarn.

In some applications, the yarn is a filament yarn, and forming the yarn includes forming the filament yarn.

In some applications, the method further includes air texturizing the filament yarn.

In some applications, the yarn is a core-spun yarn, and forming the yarn includes forming the core-spun yarn.

In some applications, incorporating the yarn in a fabric or textile includes producing the fabric/textile by interlacing lengths of the yarn.

In some applications, the fabric/textile consists substantially of only the yarn, and producing the fabric/textile includes producing the fabric/textile by interlacing lengths of substantially only the yarn.

In some applications, the yarn is a first yarn, the fabric/textile includes a mixture of the first yarn and a second yarn, and producing the fabric/textile includes producing the fabric/textile by interlacing lengths of the first yarn and lengths of the second yarn.

In some applications, producing the fabric/textile by interlacing lengths of the yarn includes producing the fabric/textile by weaving lengths of the yarn.

In some applications, producing the fabric/textile by interlacing lengths of the yarn includes producing the fabric/textile by knitting lengths of the yarn.

There is provided, in accordance with some applications, a method, including forming into fiber a mixture that includes: a polymer, and an additive molecule comprising one or more fluorinated end-groups, and forming a yarn that includes the fiber.

In some applications, the additive molecule includes (i) a polymeric backbone, and (ii) fluorinated end-groups at at least one end of the backbone; and

In some applications, the polymeric backbone includes polyurethane, and forming the mixture into the fiber includes forming, into the fiber, the mixture that includes the additive molecule that includes the polymeric backbone that includes polyurethane.

In some applications, the yarn is surgical suture that includes the fiber, and forming the yarn includes forming the surgical suture that includes the fiber.

In some applications, the method further includes incorporating the yarn in a fabric, textile, implant, and/or medical device.

In some applications, the yarn consists substantially of only the fiber, and forming the yarn includes forming the yarn from substantially only the fiber.

In some applications, the fiber is a first fiber, the yarn includes a mixture of the first fiber and a second fiber, and forming the yarn includes forming the yarn from the mixture of the first fiber and the second fiber.

In some applications, the yarn is a core-spun yarn, and forming the yarn includes forming the core-spun yarn.

In some applications, forming the yarn includes spinning the yarn.

In some applications, the method further includes cutting the fiber into staple fiber, and spinning the yarn includes spinning the staple fiber into the yarn.

In some applications, the yarn is a filament yarn, and forming the yarn includes forming the filament yarn.

In some applications, the method further includes air texturizing the filament yarn.

There is provided, in accordance with some applications, a method, including forming a yarn that includes fiber formed from a mixture of a polymer and an additive molecule that includes one or more fluorinated end-groups, and incorporating the yarn in a fabric or textile.

In some applications, the additive molecule includes (i) a polymeric backbone, and (ii) fluorinated end-groups at at least one end of the backbone.

In some applications, the polymeric backbone includes polyurethane, and forming the yarn that includes the fiber includes forming the yarn that includes the fiber formed from the mixture of the polymer and the additive molecule that includes the polymeric backbone that includes polyurethane.

In some applications, the yarn consists substantially of only the fiber, and forming the yarn includes forming the yarn from substantially only the fiber.

In some applications, the fiber is a first fiber, the yarn includes a mixture of the first fiber and a second fiber, and forming the yarn includes forming the yarn from the mixture of the first fiber and the second fiber.

In some applications, the yarn is a core-spun yarn, and forming the yarn includes forming the core-spun yarn.

In some applications, forming the yarn includes spinning the yarn.

In some applications, the method further includes cutting the fiber into staple fiber, and spinning the yarn includes spinning the staple fiber into the yarn.

In some applications, the yarn is a filament yarn, and forming the yarn includes forming the filament yarn.

In some applications, the method further includes air texturizing the filament yarn.

In some applications, incorporating the yarn in the fabric/textile includes producing a fabric/textile by interlacing lengths of the yarn.

In some applications, the fabric/textile consists substantially of only the yarn, and producing the fabric/textile includes producing the fabric/textile by interlacing lengths of substantially only the yarn.

In some applications, the yarn is a first yarn, the fabric/textile includes a mixture of the first yarn and a second yarn, and producing the fabric/textile includes producing the fabric/textile by interlacing lengths of the first yarn and lengths of the second yarn.

In some applications, producing the fabric/textile by interlacing lengths of the yarn includes producing the fabric/textile by weaving lengths of the yarn.

In some applications, producing the fabric/textile by interlacing lengths of the yarn includes producing the fabric/textile by knitting lengths of the yarn.

In some applications, the fabric/textile is incorporated into an implant and/or medical device.

There is provided, in accordance with some applications, a method, including obtaining a frame for a medical implant or device and dressing the frame with fibers that have been formed from a mixture, the mixture including: a polymer, and an additive molecule that includes one or more fluorinated end-groups.

In some applications, the additive molecule that includes (i) a polymeric backbone, and (ii) fluorinated end-groups at at least one end of the backbone.

In some applications, the polymeric backbone includes polyurethane, and dressing the frame with the fibers includes dressing the frame with the fibers that have been formed from the mixture that includes the additive molecule that includes the backbone that includes polyurethane.

In some applications, dressing the frame with the fibers includes spinning (e.g., electrospinning, jet spinning, etc.) the fibers onto the frame.

In some applications, dressing the frame with the fibers includes dressing the frame with a fabric or textile that includes the fibers.

In some applications, dressing the frame with the fabric/textile includes stitching the fabric/textile to the frame.

In some applications, dressing the frame with the fibers includes dressing only a part of the frame with the fibers.

In some applications, the part of the frame is a first part of the frame, and the method further includes dressing a second part of the frame, different from the first part of the frame, with other fibers that have not been formed from the mixture.

There is provided, in accordance with some applications, a system and/or an apparatus, including a medical implant or device that includes fibers that have been formed from a mixture, the mixture including: a polymer; and an additive molecule that includes one or more fluorinated end-groups.

In some applications, the additive molecule includes (i) a polymeric backbone, and (ii) fluorinated end-groups at at least one end of the backbone

In some applications, the polymeric backbone includes polyurethane.

In some applications, the implant or device includes a fabric or textile within which the fibers are included.

In some applications, the implant or device includes a yarn, thread, or suture within which the fibers are included.

There is provided, in accordance with some applications, a system and/or an apparatus including a fabric or textile that includes fibers that have been formed from a mixture, the mixture including: a polymer and an additive molecule that includes one or more fluorinated end-groups.

In some applications, the additive molecule that includes (i) a polymeric backbone, and (ii) fluorinated end-groups at at least one end of the backbone. In some applications, the polymeric backbone includes polyurethane.

There is provided, in accordance with some applications, a system and/or an apparatus including a surgical suture that includes fibers formed into a yarn, the fibers having been formed from a mixture including: a polymer and an additive molecule that includes one or more fluorinated end-groups.

In some applications, the additive molecule that includes (i) a polymeric backbone, and (ii) fluorinated end-groups at at least one end of the backbone. In some applications, the polymeric backbone includes polyurethane.

There is provided, in accordance with some applications, a method including: applying, to a fabric or textile, a solution that includes: a solvent, a polymer dissolved in the solvent, and an additive molecule dissolved in the solvent, the additive molecule comprising one or more fluorinated end-groups. The method can further include allowing the solvent to evaporate.

In some applications, the additive molecule includes (i) a polymeric backbone, and (ii) fluorinated end-groups at at least one end of the backbone; and

In some applications, the polymeric backbone includes polyurethane, and applying the solution to the fabric/textile includes applying, to the fabric/textile, the solution that includes the polymeric backbone that includes polyurethane.

In some applications, applying the solution to the fabric/textile includes dipping the fabric/textile into the solution.

In some applications, applying the solution to the fabric/textile includes spraying the solution onto the fabric/textile.

In some applications, the fabric/textile includes natural fibers and applying the solution to the fabric/textile includes applying the solution to the fabric/textile that includes the natural fibers.

In some applications, the fabric/textile is a blended fabric/textile and applying the solution to the fabric/textile includes applying the solution to the blended fabric/textile.

In some applications, the method further includes preparing the solution by dissolving the polymer and the additive in the solvent.

In some applications, the fabric/textile includes synthetic fibers, and applying the solution to the fabric/textile includes applying the solution to the fabric/textile that includes the synthetic fibers.

In some applications, the synthetic fibers include polyethylene terephthalate, and applying the solution to the fabric/textile includes applying the solution to the fabric/textile that includes the synthetic fibers that include polyethylene terephthalate.

In some applications, the synthetic fibers are formed from the polymer, and applying the solution to the fabric/textile includes applying the solution to the fabric/textile that includes the synthetic fibers formed from the polymer.

In some applications, the polymer is a first polymer, and the synthetic fibers are formed from a second polymer that is different to the first polymer, and applying the solution to the fabric/textile includes applying the solution to the fabric/textile that includes the synthetic fibers that are formed from the second polymer.

In some applications, the second polymer has a different degree of polymerization (DP) to the first polymer.

In some applications, the second polymer has a higher DP than the first polymer.

In some applications, the second polymer has a lower DP than the first polymer.

In some applications, the second polymer is monomerically identical to the first polymer.

In some applications, the second polymer is less soluble in the solvent than is the first polymer.

In some applications, the method further includes incorporating the fabric/textile into a medical implant or device.

In some applications, incorporating the fabric/textile into the medical implant or device includes dressing a frame of the implant or device with the fabric/textile.

In some applications, dressing the frame with the fabric/textile includes stitching the fabric/textile to the frame.

In some applications, dressing the frame with the fabric/textile includes dressing only a part of the frame with the fabric/textile.

In some applications, the part of the frame is a first part of the frame, the fabric/textile is a first fabric/textile, and the method further includes dressing a second part of the frame, different from the first part of the frame, with a second fabric/textile to which the solution has not been applied.

In some applications, incorporating the fabric/textile into the medical implant or device includes incorporating the fabric/textile into the medical implant or device subsequently to applying the solution to the fabric/textile.

In some applications, incorporating the fabric/textile into the medical implant or device includes incorporating the fabric/textile into the medical implant or device prior to applying the solution to the fabric/textile.

There is provided, in accordance with some applications, a method including applying, to a fiber, a solution that includes: a solvent, a polymer dissolved in the solvent, and an additive molecule dissolved in the solvent, the additive molecule including one or more fluorinated end-groups. The method further includes forming a residue on the fiber by allowing the solvent to evaporate, the residue including the polymer and the additive molecule.

In some applications, the method further includes concentrating the additive molecule toward a surface of the residue by heating the residue and, subsequently, allowing the residue to cool.

In some applications, the additive molecule includes (i) a polymeric backbone, and (ii) fluorinated end-groups at at least one end of the backbone.

In some applications, heating the residue includes heating the residue by at least 50 degrees C.

In some applications, heating the residue includes heating the residue to a temperature that is greater than a melting temperature of the polymer and lower than a melting temperature of the fiber.

In some applications, the polymeric backbone includes polyurethane, and applying the solution to the fiber includes applying, to the fiber, the solution that includes the polymeric backbone that includes polyurethane.

In some applications, the method further includes incorporating the fiber in a yarn, fabric, textile, implant, and/or medical device.

In some applications, incorporating the fiber in the yarn, fabric, textile, implant, and/or medical device includes incorporating the fiber in the yarn, fabric, textile, implant, and/or medical device subsequently to concentrating the additive molecule toward the surface of the residue by heating the residue.

In some applications, incorporating the fiber in the yarn, fabric, textile, implant, and/or medical device includes incorporating the fiber in the yarn, fabric, textile, implant, and/or medical device prior to concentrating the additive molecule toward the surface of the residue by heating the residue.

In some applications, applying the solution to the fiber includes applying the solution to the fiber prior to incorporating the fiber in the yarn, fabric, textile, implant, and/or medical device.

In some applications, applying the solution to the fiber includes applying the solution to the fiber subsequently to incorporating the fiber in the yarn, fabric, textile, implant, and/or medical device.

In some applications, heating the residue includes heating the residue for no more than 1 minute. In some applications, heating the residue includes heating the residue for no more than 30 seconds. In some applications, heating the residue includes heating the residue for no more than 10 seconds. In some applications, heating the residue includes heating the residue for no more than 5 seconds. In some applications, heating the residue includes heating the residue for no more than 1 second.

In some applications, the fiber is a synthetic fiber, and applying the solution to the fiber includes applying the solution to the synthetic fiber.

In some applications, the synthetic fiber includes polyethylene terephthalate, and applying the solution to the synthetic fiber includes applying the solution to the synthetic fiber that includes polyethylene terephthalate.

In some applications, the synthetic fiber is formed from the polymer, and applying the solution to the synthetic fiber includes applying the solution to the synthetic fiber formed from the polymer.

In some applications, the polymer is a first polymer, the synthetic fiber is formed from a second polymer that is different from the first polymer, and applying the solution to the synthetic fiber includes applying the solution to the synthetic fiber formed from the second polymer.

In some applications, the second polymer has a different degree of polymerization (DP) than that of the first polymer.

In some applications, the second polymer has a higher DP than that of the first polymer.

In some applications, the second polymer has a lower DP than that of the first polymer.

In some applications, the second polymer is monomerically identical to the first polymer.

In some applications, the second polymer is less soluble in the solvent than is the first polymer.

In some applications, the second polymer has a different melting temperature than that of the first polymer. In some applications, the second polymer has a higher melting temperature than that of the first polymer. In some applications, the second polymer has a lower melting temperature than that of the first polymer.

In some applications, the second polymer has a different glass-transition temperature than that of the first polymer. In some applications, the second polymer has a higher glass-transition temperature than that of the first polymer. In some applications, the second polymer has a lower glass-transition temperature than that of the first polymer.

There is provided, in accordance with some applications, a method including applying, to a fiber, a solution that includes: a solvent, a polymer dissolved in the solvent, and an additive molecule dissolved in the solvent, the additive molecule including one or more fluorinated end-groups. The method further includes allowing the solvent to evaporate. The method can further include forming a yarn that includes the fiber.

In some applications, the additive molecule includes (i) a polymeric backbone, and (ii) fluorinated end-groups at at least one end of the backbone.

In some applications, the polymeric backbone includes polyurethane, and applying the solution to the fiber includes applying, to the fiber, the solution that includes the additive molecule that includes the backbone that includes polyurethane.

In some applications, the method further includes incorporating the yarn into a fabric or textile.

In some applications, the yarn is a medical suture, and forming the yarn includes forming the medical suture.

There is provided, in accordance with some applications, a method including applying, to a yarn, a solution that includes: a solvent, a polymer dissolved in the solvent, and an additive molecule dissolved in the solvent, the additive molecule including one or more fluorinated end-groups. The method further includes allowing the solvent to evaporate.

In some applications, the additive molecule includes (i) a polymeric backbone, and (ii) fluorinated end-groups at at least one end of the backbone.

In some applications, the polymeric backbone includes polyurethane, and applying the solution to the yarn includes applying, to the yarn, the solution that includes the additive molecule that includes the backbone that includes polyurethane.

In some applications, the method further includes incorporating the yarn into a fabric or textile.

In some applications, the yarn is a medical suture, and applying the solution to the yarn includes applying the solution to the medical suture.

There is provided, in accordance with some applications, a method including applying, to a fiber, a solution that includes: a solvent, a polymer dissolved in the solvent, and an additive molecule dissolved in the solvent, the additive molecule including one or more fluorinated end-groups. The method further includes allowing the solvent to evaporate and forming a fabric or textile that includes the fiber.

In some applications, the additive molecule includes (i) a polymeric backbone, and (ii) fluorinated end-groups at at least one end of the backbone

In some applications, the polymeric backbone includes polyurethane, and applying the solution to the fiber includes applying, to the fiber, the solution that includes the additive molecule that includes the backbone that includes polyurethane.

In some applications, allowing the solvent to evaporate includes forming a residue on the fiber by allowing the solvent to evaporate, the residue including the polymer and the additive molecule. In some applications, the method further includes concentrating the additive molecule toward a surface of the residue by heating the residue and, subsequently, allowing the residue to cool.

In some applications, forming the fabric/textile includes forming the fabric/textile subsequently to concentrating the additive molecule toward the surface of the residue by heating the residue.

In some applications, forming the fabric/textile includes forming the fabric/textile prior to concentrating the additive molecule toward the surface of the residue by heating the residue.

There is provided, in accordance with some applications, a system and/or an apparatus, including a medical implant or medical device that includes fibers to which a solution has been applied, the solution including: a solvent; a polymer dissolved in the solvent, and an additive molecule dissolved in the solvent, the additive molecule including one or more fluorinated end-groups.

In some applications, the additive molecule includes (i) a polymeric backbone, and (ii) fluorinated end-groups at at least one end of the backbone.

In some applications, the medical implant or device includes stitches of a thread that includes the fibers.

In some applications, the polymeric backbone includes polyurethane.

In some applications, the fibers are first fibers, the solution is a first solution, and the medical implant or device further includes second fibers to which a second solution has been applied, the second solution including: a solvent and a fluorinated polyphosphazene.

In some applications, the medical implant or device includes a fabric or textile that includes the fibers.

In some applications, the fibers are first fibers, the solution is a first solution, the fabric/textile is a first fabric/textile, and the medical implant or device further includes a second fabric/textile that includes second fibers to which a second solution has been applied, the second solution including: a solvent and a fluorinated polyphosphazene.

In some applications, the fibers are first fibers, the solution is a first solution, and the fabric/textile further includes second fibers to which a second solution has been applied, the second solution including: a solvent and a fluorinated polyphosphazene.

In some applications, the system and/or apparatus is for use at a valve of a heart of a subject, the valve having a first leaflet and at least one opposing leaflet, the heart having a chamber upstream of the valve.

In some applications, the implant or device includes an interface (e.g., an anchor mount, multiple anchor mounts, a hub(s), a connection(s), etc.) and a flexible coaptation portion (e.g., a leaf, sheet, mesh, wire form, membrane, spacer, expandable member, inflatable member, balloon, foam, combination of two or more of these, etc.), coupled to the interface.

In some applications, the implant or device includes a flexible wire frame. In some applications, the flexible wire frame is shaped as a loop (or multiple loops or struts) that defines an aperture (or multiple apertures) therethrough and a fabric or textile, coupled to the wire and covering at least part of the aperture(s), the fabric/textile including the fibers.

In some applications, the implant or device includes an anchor, and the system and/or apparatus further includes a delivery system including: a shaft, configured to, via engagement with the interface, position the implant or device in a position in which the interface is at a site in the heart, the coaptation portion (e.g., a leaf, sheet, mesh, wire form, membrane, spacer, expandable member, inflatable member, balloon, foam, combination of two or more of these, etc.) extends over the first leaflet toward the opposing leaflet, and the contact face faces the first leaflet; and an anchor driver, configured to secure the implant or device in the position by anchoring the interface to tissue of the heart by driving the anchor into the tissue. The coaptation portion can be configured to prevent or inhibit flail or prolapse of the first leaflet and maintain good coaptation with the opposing leaflet (i.e., allow the valve to close with little or no regurgitation).

In some applications, the wire is arranged to define the loop as teardrop-shaped.

In some applications, the coaptation portion has an open part at which the aperture is not covered by the fabric/textile.

In some applications, the coaptation portion has a root that is coupled to the interface, and a tip at an opposite end of the coaptation portion from the root, and the fabric/textile is disposed between the open part and the tip.

In some applications, the implant or device includes an annuloplasty structure.

In some applications, the annuloplasty structure includes a textile or fabric sleeve that includes the fibers.

In some applications, the implant or device includes a prosthetic heart valve.

In some applications, the prosthetic heart valve includes a frame, and a textile or fabric sheet that includes the fibers, the fabric/textile sheet disposed over at least part of the frame.

In some applications, the prosthetic heart valve includes a thread that stitches components of the prosthetic heart valve together, and the thread includes the fibers.

In some applications, one of the components is a frame, and the thread stitches another of the components to the frame.

In some applications, the other of the components is a textile or fabric sheet.

In some applications, the other of the components is a prosthetic leaflet.

There is provided, in accordance with some applications, a system and/or an apparatus, including a fabric/textile to which a solution has been applied, the solution including: a solvent, a polymer dissolved in the solvent, and an additive molecule, dissolved in the solvent, the additive molecule including one or more fluorinated end-groups.

In some applications, the additive molecule including (i) a polymeric backbone, and (ii) fluorinated end-groups at at least one end of the backbone. In some applications, the polymeric backbone includes polyurethane.

There is provided, in accordance with some applications, a system and/or an apparatus, including a surgical suture that includes fibers formed into a yarn, the fibers having had a solution applied thereto, the solution including: a solvent, a polymer dissolved in the solvent, and an additive molecule, dissolved in the solvent, the additive molecule includes one or more fluorinated end-groups.

In some applications, the additive molecule, dissolved in the solvent, the additive molecule including (i) a polymeric backbone, and (ii) fluorinated end-groups at at least one end of the backbone. In some applications, the polymeric backbone includes polyurethane.

There is provided, in accordance with some applications, a system and/or an apparatus, including a prosthetic heart valve that includes a frame and prosthetic leaflets supported by the frame, the prosthetic leaflets having had a solution applied thereto, the solution including: a solvent, a polymer dissolved in the solvent, and an additive molecule dissolved in the solvent, the additive molecule including one or more fluorinated end-groups.

In some applications, the additive molecule, dissolved in the solvent, the additive molecule including (i) a polymeric backbone, and (ii) fluorinated end-groups at at least one end of the backbone. In some applications, the polymeric backbone includes polyurethane.

There is provided, in accordance with some applications, a method including applying, to a fabric or textile, a solution that includes: a solvent and a fluorinated polyphosphazene. The method can further include allowing the solvent to evaporate.

In some applications, applying the solution to the fabric/textile includes dipping the fabric/textile into the solution.

In some applications, applying the solution to the fabric/textile includes spraying the solution onto the fabric/textile.

In some applications, the fabric/textile includes natural fibers, and applying the solution to the fabric/textile includes applying the solution to the fabric/textile that includes the natural fibers.

In some applications, the fabric/textile is a blended fabric/textile, and applying the solution to the fabric/textile includes applying the solution to the blended fabric/textile.

In some applications, the method further includes preparing the solution by dissolving the fluorinated polyphosphazene in the solvent.

In some applications, the fabric/textile includes synthetic fibers, and applying the solution to the fabric/textile includes applying the solution to the fabric/textile that includes the synthetic fibers.

In some applications, the synthetic fibers are formed from nylon, and applying the solution to the fabric/textile includes applying the solution to the fabric/textile that includes the synthetic fibers formed from nylon.

In some applications, the synthetic fibers are formed from an elastomer, and applying the solution to the fabric/textile includes applying the solution to the fabric/textile that includes the synthetic fibers formed from the elastomer.

In some applications, the synthetic fibers are formed from a polyester, and applying the solution to the fabric/textile includes applying the solution to the fabric/textile that includes the synthetic fibers formed from the polyester.

In some applications, the polyester is polyethylene terephthalate, and applying the solution to the fabric/textile includes applying the solution to the fabric/textile that includes the synthetic fibers formed from polyethylene terephthalate.

In some applications, the synthetic fibers are formed from a thermoplastic polymer, such as thermoplastic polyurethane, and applying the solution to the fabric/textile includes applying the solution to the fabric/textile that includes the synthetic fibers formed from the thermoplastic polymer.

In some applications, the method further includes incorporating the fabric/textile into a medical implant or device.

In some applications, incorporating the fabric/textile into the medical implant or device includes dressing a frame of the implant or device with the fabric/textile.

In some applications, dressing the frame with the fabric/textile includes stitching the fabric/textile to the frame.

In some applications, dressing the frame with the fabric/textile includes dressing only a part of the frame with the fabric/textile.

In some applications, the part of the frame is a first part of the frame, the fabric/textile is a first fabric/textile, and the method further includes dressing a second part of the frame, different from the first part of the frame, with a second fabric/textile to which the solution has not been applied.

In some applications, incorporating the fabric/textile into the medical implant or device includes incorporating the fabric/textile into the medical implant or device subsequently to applying the solution to the fabric/textile.

In some applications, incorporating the fabric/textile into the medical implant or device includes incorporating the fabric/textile into the medical implant or device prior to applying the solution to the fabric/textile.

There is provided, in accordance with some applications, a method, including: applying, to a fiber, a solution that includes: a solvent and a fluorinated polyphosphazene. The method can further include allowing the solvent to evaporate and forming a yarn that includes the fiber.

In some applications, the method further includes incorporating the yarn into a fabric or textile.

In some applications, the yarn is a medical suture, and forming the yarn includes forming the medical suture.

There is provided, in accordance with some applications, a method, including: applying, to a yarn, a solution that includes: a solvent and a fluorinated polyphosphazene. The method can further include allowing the solvent to evaporate.

In some applications, the method further includes incorporating the yarn into a fabric or textile.

In some applications, the yarn is a medical suture, and applying the solution to the yarn includes applying the solution to the medical suture.

There is provided, in accordance with some applications, a system and/or an apparatus, including a medical implant or device that includes fibers to which a solution has been applied, the solution including: a solvent and a fluorinated polyphosphazene.

In some applications, the medical implant or device includes stitches of a thread that includes the fibers.

In some applications, the fibers are first fibers, the solution is a first solution, and the medical implant or device further includes second fibers to which a second solution has been applied, the second solution including: a second solvent, a polymer dissolved in the second solvent, and an additive molecule dissolved in the second solvent, the additive molecule including one or more fluorinated end-groups. In some applications, the additive molecule includes (i) a polymeric backbone, and (ii) fluorinated end-groups at at least one end of the backbone.

In some applications, the medical implant or device includes a thread that includes the fibers.

In some applications, the medical implant or device includes a fabric or textile that includes the fibers.

In some applications, the fibers are first fibers, the solution is a first solution, the fabric/textile is a first fabric/textile, and the medical implant or device further includes a second fabric/textile that includes second fibers to which a second solution has been applied, the second solution including: a second solvent, a polymer dissolved in the second solvent, and an additive molecule dissolved in the second solvent, the additive molecule including one or more fluorinated end-groups. In some applications, the additive molecule including (i) a polymeric backbone, and (ii) fluorinated end-groups at at least one end of the backbone.

In some applications, the fibers are first fibers, the solution is a first solution, and the fabric/textile further includes second fibers to which a second solution has been applied, the second solution including: a second solvent, a polymer, dissolved in the second solvent, and an additive molecule dissolved in the second solvent, the additive molecule including one or more fluorinated end-groups. In some applications, the additive molecule including (i) a polymeric backbone, and (ii) fluorinated end-groups at at least one end of the backbone.

In some applications, the system and/or apparatus is for use at a valve of a heart of a subject, the valve having a first leaflet and at least one opposing leaflet, the heart having a chamber upstream of the valve.

In some applications, the implant or device includes: an interface, a flexible coaptation portion, coupled to the interface, and includes a flexible wire (which can be shaped as a loop that defines an aperture therethrough), and includes a fabric/textile, coupled to the wire and covering at least part of the aperture, the fabric/textile including the fibers.

In some applications, the implant or device includes an anchor.

In some applications, the system and/or apparatus further includes a delivery system including a shaft configured to, via engagement with the interface, position the implant or device in a position in which the interface is at a site in the heart and the coaptation portion extends over the first leaflet toward the opposing leaflet, and including an anchor driver, configured to secure the implant or device in the position by anchoring the interface to tissue of the heart by driving the anchor into the tissue. The coaptation portion can extend over the first leaflet to prevent or inhibit the first leaflet from flailing or prolapsing and help maintain good coaptation between the leaflets.

In some applications, the wire is arranged to define the loop as teardrop-shaped.

In some applications, the coaptation portion has an open part at which the aperture is not covered by the fabric/textile.

In some applications, the coaptation portion or leaf has a root that is coupled to the interface, and a tip at an opposite end of the coaptation portion or leaf from the root, and the fabric/textile is disposed between the open part and the tip.

In some applications, the implant or device includes an annuloplasty structure.

In some applications, the annuloplasty structure includes a fabric/textile sleeve that includes the fibers.

In some applications, the implant or device includes a prosthetic heart valve.

In some applications, the prosthetic heart valve includes a frame, and a fabric/textile sheet that includes the fibers, the fabric/textile sheet disposed over at least part of the frame.

In some applications, the prosthetic heart valve includes a thread that stitches components of the prosthetic heart valve together, and the thread includes the fibers.

In some applications, one of the components is a frame, and the thread stitches another of the components to the frame.

In some applications, the other of the components is a fabric/textile sheet.

In some applications, the other of the components is a prosthetic leaflet.

There is provided, in accordance with some applications, a system and/or an apparatus, including a surgical suture including fibers formed into a yarn, the fibers having had a solution applied thereto, the solution including: a solvent and a fluorinated polyphosphazene.

There is provided, in accordance with some applications, a system and/or an apparatus, including a prosthetic heart valve that includes: a frame and prosthetic leaflets, supported by the frame. The prosthetic heart valve having had a solution applied to one or more portions thereof, the solution including a solvent and a fluorinated polyphosphazene.

In some applications, the prosthetic leaflets had the solution applied thereto.

In some applications, the frame had the solution applied thereto.

In some applications, a fabric or textile attached to the frame had the solution applied thereto.

There is provided, in accordance with some applications, a system and/or an apparatus, including a fabric/textile to which a solution has been applied, the solution including a solvent and a fluorinated polyphosphazene.

There is provided, in accordance with some applications, a method of forming a stabilized fabric, the method including weaving a fabric from multiple strands of implantable yarn, each strand of implantable yarn at least partially coated with a thermoplastic material. The method can further include heating junction points in the fabric to fix intersecting strands of yarn to each other, thereby to reinforce the structure of the fabric.

In some applications, the method can further include at least partially coating each of the multiple strands of yarn with the thermoplastic material.

In some applications, the multiple strands of implantable yarn include polyethylene.

In some applications, the thermoplastic material includes thermoplastic polyurethane.

In some applications, the thermoplastic material includes fluorinated ethylene propylene. In some applications, the thermoplastic material includes ultrahigh molecular weight polyethylene. In some applications, the thermoplastic material includes expanded polytetrafluoroethylene.

In some applications, the at least partially coating includes coating the entirety of the exterior of the multiple strands of the implantable yarn with the thermoplastic material.

In some applications, the coating includes forming an implantable yarn from a plurality of implantable fibers, and coating the formed implantable yarn with the thermoplastic material.

In some applications, the coating of the formed implantable yarn includes co-extruding the formed implantable yarn and the thermoplastic material.

In some applications, the coating includes coating a plurality of implantable fibers with the thermoplastic material, and forming the implantable yarn from the plurality of implantable fibers, following coating thereof.

In some applications, the at least partially coating of the plurality of implantable fibers includes co-extruding the plurality of implantable fibers and the thermoplastic material.

In some applications, the at least partially coating includes spinning a strand formed of the thermoplastic material about a core formed of the implantable yarn.

In some applications, the at least partially coating includes twisting together at least one strand of the implantable yarn and at least one strand of the thermoplastic material, when they are disposed parallel to one another.

In some applications, the weaving includes weaving a leno fabric defining a plurality of windows. In some applications, the greatest dimension of the plurality of windows is not greater than 2 mm. In some applications, the greatest dimension of the plurality of windows is not greater than 1.8 mm. In some applications, the greatest dimension of the plurality of windows is not greater than 1.5 mm. In some applications, the greatest dimension of the plurality of windows is not greater than 1.2 mm. In some applications, the greatest dimension of the plurality of windows is not greater than 1 mm.

In some applications, following the heating, the fabric has a thickness not greater than 80 μm. In some applications, following the heating, the fabric has a thickness not greater than 75 μm. In some applications, following the heating, the fabric has a thickness not greater than 70 μm. In some applications, following the heating, the fabric has a thickness not greater than 65 μm.

In some applications, the heating includes heat pressing the fabric to heat the junction points.

In some applications, the heating includes locally heating each of the junction points.

In some applications, the method can include, following the heating, laminating at least one side of the fabric.

In some applications, the laminating includes laminating only one side of the fabric.

In some applications, the laminating includes laminating both sides of the fabric.

In some applications, the laminating includes laminating the fabric in a pattern to form laminated regions and non-laminated regions.

There is provided, in accordance with some applications, a stabilized fabric, including a woven structure formed from a plurality of strands of yarn, each including an implantable yarn, the exterior of which is at least partially coated with a thermoplastic material. At least one junction point between some of the plurality of strands of yarn in the woven structure can have been heated to retain the relative positioning of the some of the plurality of strands of yarn.

In some applications, the implantable yarn includes polyethylene.

In some applications, the thermoplastic material includes thermoplastic polyurethane.

In some applications, the thermoplastic material includes fluorinated ethylene propylene. In some applications, the thermoplastic material includes ultrahigh molecular weight polyethylene. In some applications, the thermoplastic material includes expanded polytetrafluoroethylene.

In some applications, the exterior of the implantable yarn is entirely coated with the thermoplastic material.

In some applications, the implantable yarn is formed of a plurality of implantable fibers, the exterior of each of the implantable fibers being fully coated with the thermoplastic material.

In some applications, the yarn includes a core of the implantable yarn, about which is spun a strand of the thermoplastic material.

In some applications, the yarn includes a strand of the implantable yarn twisted together with a strand of the thermoplastic material.

In some applications, the woven structure includes a leno fabric structure defining a plurality of windows. In some applications, the greatest dimension of the plurality of windows is not greater than 2 mm. In some applications, the greatest dimension of the plurality of windows is not greater than 1.8 mm. In some applications, the greatest dimension of the plurality of windows is not greater than 1.5 mm. In some applications, the greatest dimension of the plurality of windows is not greater than 1.2 mm. In some applications, the greatest dimension of the plurality of windows is not greater than 1 mm.

In some applications, the fabric has a thickness not greater than 80 μm. In some applications, the fabric has a thickness not greater than 75 μm. In some applications, the fabric has a thickness not greater than 70 μm. In some applications, the fabric has a thickness not greater than 65 μm.

In some applications, the fabric is laminated on at least one side thereof.

In some applications, the fabric is laminated only on one side thereof.

In some applications, the fabric is laminated on both sides thereof.

In some applications, the fabric is laminated in a pattern which forms laminated regions and non-laminated regions.

There is provided, in accordance with some applications, a method of forming a stabilized fabric, the method including forming a mesh fabric from strands of an implantable yarn and stabilizing the mesh fabric by coating at least one side of the mesh fabric with a thermoplastic polymer.

In some applications, the forming of the mesh fabric includes forming the mesh fabric from an implantable yarn including polyethylene.

In some applications, the forming of the mesh fabric includes weaving a leno fabric defining a plurality of windows as the mesh fabric. In some applications, the greatest dimension of the plurality of windows is not greater than 2 mm. In some applications, the greatest dimension of the plurality of windows is not greater than 1.8 mm. In some applications, the greatest dimension of the plurality of windows is not greater than 1.5 mm. In some applications, the greatest dimension of the plurality of windows is not greater than 1.2 mm. In some applications, the greatest dimension of the plurality of windows is not greater than 1 mm.

In some applications, the forming of the mesh fabric includes knitting a mesh knit as the mesh fabric.

In some applications, the coating includes coating the at least one side of the mesh fabric with the thermoplastic polymer. In some applications, the thermoplastic polymer includes thermoplastic polyurethane. In some applications, the thermoplastic polymer includes fluorinated ethylene propylene. In some applications, the thermoplastic polymer includes ultrahigh molecular weight polyethylene. In some applications, the thermoplastic polymer includes expanded polytetrafluoroethylene.

In some applications, the coating includes spray coating the mesh fabric with a solution including the thermoplastic polymer.

In some applications, the coating includes dip coating the mesh fabric with a solution including the thermoplastic polymer.

In some applications, the coating includes forming the solution including the thermoplastic polymer and a solvent. In some applications, the solvent includes tetrahydrofuran. In some applications, the solvent includes dimethylacetamide. In some applications, the solvent includes acetone.

In some applications, the coating includes, following application of the solution onto the mesh fabric, allowing the solvent to evaporate, washing and drying the mesh fabric, and heat pressing the mesh fabric.

In some applications, the coating includes laminating the mesh fabric.

In some applications, the laminating includes laminating the fabric in a pattern to form laminated regions and non-laminated regions.

In some applications, the coating includes coating only one side of the mesh fabric.

In some applications, the coating includes coating both sides of the mesh fabric.

In some applications, following the coating, the fabric has a thickness not greater than 80 μm. In some applications, following the coating, the fabric has a thickness not greater than 75 μm. In some applications, following the coating, the fabric has a thickness not greater than 70 μm. In some applications, following the coating, the fabric has a thickness not greater than 65 μm.

In some applications, the method can include, following the coating, at least one of scouring and sterilizing the mesh fabric.

There is provided in accordance with some applications a stabilized fabric including a mesh fabric formed from strands of an implantable yarn, at least one side of the mesh fabric being coated with a thermoplastic polymer.

In some applications, the implantable yarn includes polyethylene.

In some applications, the mesh fabric includes a leno fabric defining a plurality of windows. In some applications, the greatest dimension of the plurality of windows is not greater than 2 mm. In some applications, the greatest dimension of the plurality of windows is not greater than 1.8 mm. In some applications, the greatest dimension of the plurality of windows is not greater than 1.5 mm. In some applications, the greatest dimension of the plurality of windows is not greater than 1.2 mm. In some applications, the greatest dimension of the plurality of windows is not greater than 1 mm.

In some applications, the mesh fabric includes a mesh knit.

In some applications, the thermoplastic polymer includes thermoplastic polyurethane.

In some applications, the thermoplastic polymer includes fluorinated ethylene propylene. In some applications, the thermoplastic polymer includes ultrahigh molecular weight polyethylene. In some applications, the thermoplastic polymer includes expanded polytetrafluoroethylene.

In some applications, the thermoplastic polymer forms a laminate over the at least one side of the mesh fabric.

In some applications, the laminate coating is patterned and defines laminated regions and non-laminated regions.

In some applications, the mesh fabric is coated only on one side thereof.

In some applications, the mesh fabric is coated on both sides thereof.

In some applications, the fabric has a thickness not greater than 80 μm. In some applications, the fabric has a thickness not greater than 75 μm. In some applications, the fabric has a thickness not greater than 70 μm. In some applications, the fabric has a thickness not greater than 65 μm.

There is provided in accordance with some applications a method of forming a circumferential length of fabric, the method including stretching lengths of multifilament yarn as warp strands.

In some applications, the method further includes weaving a fabric by passing a length of a monofilament yarn between the warp strands in a first direction, and then passing the length of the monofilament yarn between the warp strands in an opposing direction, such that the monofilament yarn forms weft strands attached by folds of the monofilament yarn at lateral edges of the fabric.

In some applications, the method further includes attaching opposing ends of each of the warp strands to form a circumferential structure having the warp strands forming circumferences of the circumferential structure and having the weft strands extending perpendicularly to the circumferences, with the folds of the monofilament yarn disposed at circumferential ends of the circumferential structure.

In some applications, the weaving includes weaving the monofilament yarn together with additional lengths of multifilament yarn as the weft strands.

In some applications, the weaving includes weaving a nitinol fiber as, or with, the monofilament yarn.

In some applications, the method can include scouring the fabric.

In some applications, the method can include heat pressing the fabric.

In some applications, the method can include sterilizing the fabric.

In some applications, the method can include, prior to the attaching, at least partially laminating at least one side of the fabric with a thermoplastic polymer.

In some applications, the at least partially laminating includes laminating the fabric in a pattern to form laminated regions and non-laminated regions.

In some applications, the laminating in a pattern includes laminating in a pattern such that the laminated regions are not parallel to the warp strands or the weft strands. In some applications, the laminating in a pattern includes laminating such that the laminated regions form a trellis structure and the non-laminated regions are rhombus shaped.

In some applications, the laminating includes laminating using a thermoplastic polymer.

In some applications, the thermoplastic polymer includes thermoplastic polyurethane. In some applications, the thermoplastic polymer includes fluorinated ethylene propylene. In some applications, the thermoplastic polymer includes ultrahigh molecular weight polyethylene. In some applications, the thermoplastic polymer includes expanded polytetrafluoroethylene.

In some applications, the attaching is such that a laminated surface of the fabric is disposed on an interior side of the circumferential structure.

There is provided, in accordance with some applications, a method of forming a medical implant, the method including forming a circumferential length of fabric according to some applications described herein, and attaching the circumferential length of fabric to an implantable frame, such that the warp strands of the fabric extend about a circumference of the implantable frame.

There is provided, in accordance with some applications, an apparatus including lengths of multifilament yarn as warp strands, and a length of a monofilament yarn stretched between the warp strands in a first direction, and then folded and stretched between the warp strands in an opposing direction to form a fabric, such that the monofilament yarn forms weft strands attached by folds of the monofilament yarn at lateral edges of the fabric.

In some applications, opposing ends of each of the warp strands being attached to one another to form a circumferential structure of the fabric, having the warp strands forming circumferences of the circumferential structure and having the weft strands extending perpendicularly to the circumferences, with the folds of the monofilament yarn disposed at circumferential ends of the circumferential structure.

In some applications, the weft strands include the monofilament yarn together with additional lengths of multifilament yarn.

In some applications, the weft strands include a nitinol fiber as, or with, the monofilament yarn.

In some applications, the fabric is at least partially laminated on at least one side thereof with a thermoplastic polymer.

In some applications, the fabric is laminated in a pattern to form laminated regions and non-laminated regions.

In some applications, in the pattern, the laminated regions are not parallel to the warp strands or the weft strands.

In some applications, in the pattern, the laminated regions form a trellis structure and the non-laminated regions are rhombus shaped.

In some applications, the thermoplastic polymer includes thermoplastic polyurethane.

In some applications, the thermoplastic polymer includes fluorinated ethylene propylene. In some applications, the thermoplastic polymer includes ultrahigh molecular weight polyethylene. In some applications, the thermoplastic polymer includes expanded polytetrafluoroethylene.

In some applications, the laminated surface of the fabric is disposed on an interior side of the circumferential structure.

There is provided, in accordance with some applications, a medical implant, including an implantable frame, and a circumferential structure according to the apparatus of some applications described herein, attached about a circumference of the implantable frame.

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

### BRIEF DESCRIPTION OF THE DRAWINGS

FIGS. 1A-C, 2, and 3 are schematic illustrations, and flowcharts illustrating at least some steps, of techniques for manufacturing fibers, yarns, fabrics, and/or textiles in accordance with some applications;

FIGS. 4A-C and 5-8 are schematic illustrations, and flowcharts illustrating at least some steps, of techniques for manufacturing fibers, yarns, fabrics, and/or textiles, in accordance with some applications;

FIGS. 9A-C and 10-13 are schematic illustrations, and flowcharts illustrating at least some steps, of techniques for manufacturing fibers, yarns, fabrics, and/or textiles, in accordance with some applications;

FIGS. 14, 15, and 16 are schematic illustrations of example medical devices that comprise one or more of the above fibers, yarns, fabrics, and/or textiles, in accordance with some applications;

FIG. 17 is a schematic illustration illustrating at least some steps of a technique for manufacturing a stabilized fabric in accordance with some applications;

FIG. 18 is a schematic illustration illustrating at least some steps of a technique for manufacturing a stabilized laminated fabric in accordance with some applications; and

FIG. 19 is a schematic illustration illustrating at least some steps of a technique for manufacturing a stabilized fabric, and for using the manufactured fabric in an implantable device, in accordance with some applications.

### DETAILED DESCRIPTION

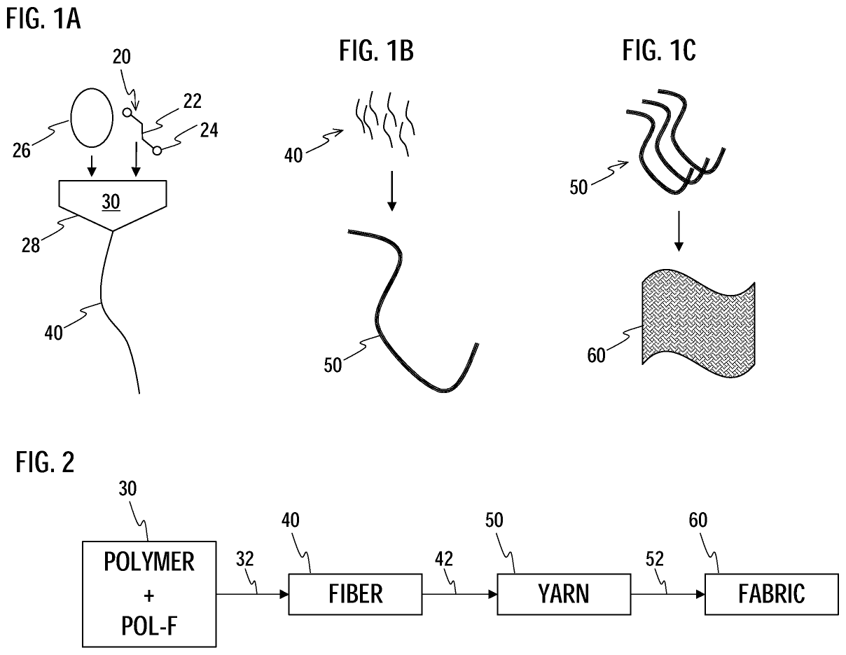

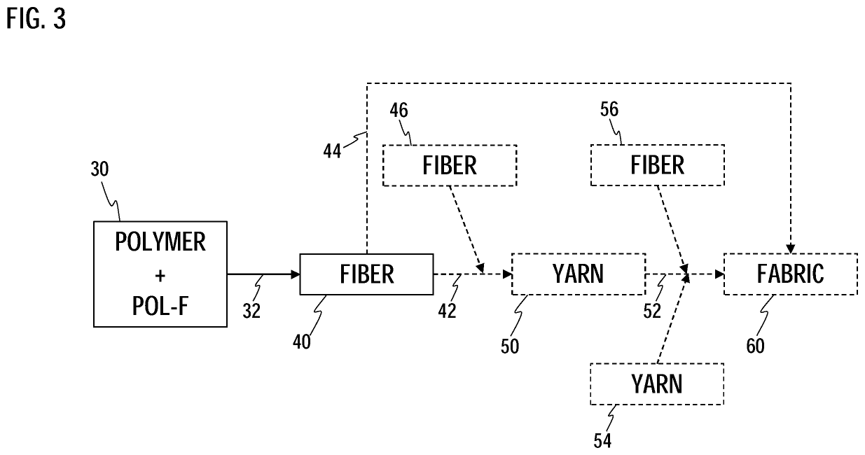

Reference is made to FIGS. 1A-C, 2, and 3, which are schematic illustrations, and flowcharts illustrating at least some steps, of techniques for manufacturing fibers, yarns, textiles, and/or fabrics, in accordance with some applications. These techniques comprise incorporation of an additive molecule 20 into synthetic fibers, and thereby also into any yarns, textiles, and/or fabrics into which the synthetic fibers may be incorporated. The term “yarn” as used herein encompasses a variety of elongate components like yarn, sutures, thread, string, etc.

For some applications, additive molecule 20 has a polymeric backbone 22 with one or more fluorinated end-groups 24 at at least one end of the backbone. In some instances herein, including in the figures, because molecule 20 can comprise a polymeric backbone 22 with fluorinated end-groups 24, molecule 20 is referred to as POL-F.

For some applications, additive molecule 20 (and any of the other additive molecules described for any of the various applications herein) can be or comprise a surface modifying macromolecule. For some applications, molecule 20 can be or comprise a low molecular weight fluoro-oligomer. For some applications, molecule 20 can be or comprise a polyphosphazene molecule. For some applications, molecule 20 can be or comprise a fluoropolymer molecule. For some applications, molecule 20 can be or comprise a Polyzene-F (PzF) molecule. For some applications, molecule 20 comprises a combination of two or more of the foregoing.

For some applications, backbone 22 comprises polyurethane (e.g., consists substantially of polyurethane).

Alternatively or additionally, backbone 22 can comprise (e.g., can consist substantially of) a different polymer, such as polyethylene terephthalate, polyurethane urea, a polyurethane block copolymer, polycarbonate urethane, a polyester urethane, a polysiloxane urethane, a polysiloxane urea, a polyisobutylene urethane urea, a polyethylenebutylene urethane urea, a polyester, a polyether block amide, or a nylon.

For some applications, backbone 22 is a homopolymer. For some applications, backbone 22 is a copolymer (e.g., a block copolymer).

From a mixture 30, one or more fibers 40 are formed, e.g., by extrusion, spinning (e.g., wet spinning, dry spinning, melt spinning, gel spinning, jet spinning, electrospinning, etc.), or drawing (FIG. 1A). Mixture 30 comprises a polymer 26 and molecule 20. For some applications, mixture 30 is formed just prior to fiber formation, e.g., by separately introducing polymer 26 and molecule 20 into a device 28 (e.g., a hopper thereof) that will be used to form the fibers, such as an extrusion device or a spinning device. For some applications, mixture 30 is provided pre-prepared (e.g., pre-mixed). Optional techniques for preparing mixture 30 can include, but are not limited to, heat-induced melt mixing, solvent-induced mixing, compounding, and/or extrusion.

The inclusion of molecule 20 in mixture 30 results in molecule 20 being incorporated into fibers 40. It is hypothesized that the incorporation of molecule 20 into fibers 40 confers advantageous properties on the fibers (and on yarns and fabrics/textiles in which the fibers are incorporated) compared to similar fibers that do not contain molecule 20. For example, it is hypothesized that, for applications in which fibers 40 are used within the human body (e.g., as described in more detail hereinbelow), the presence of molecule 20 in fibers 40 advantageously inhibits thrombogenesis, fouling, and bacterial adherence and growth. For example, molecule 20 can be configured to promote a thin layer of endothelium to promote a smooth benign surface for native anatomy to interact with, while preventing over thickening.

Polymer 26 is often the major component (e.g., the structural component) of fibers 40, and may therefore be referred to as the base polymer. For some applications, polymer 26 comprises a homopolymer. For some applications, polymer 26 comprises a copolymer. It is to be noted that, although polymer 26 is referred to in the singular, for some applications polymer 26 may refer to a blend of more than one polymer.

In some applications, mixture 30 comprises molecule 20 at at least 1 percent and/or no more than 5 percent by mass. For example, mixture 30 can comprise molecule 20 at 1-5 percent, e.g., 2-5 percent, e.g., 2-4 percent (such as 3-4 percent) or 3-5 percent (such as 4-5 percent), or 1-4 percent (e.g., 1-3 percent, such as 1-2 percent or 2-3 percent) by mass.

For some applications, polymer 26 comprises a polyester. For example, polymer 26 can comprise a polyethylene terephthalate homopolymer or copolymer. For some applications, polymer 26 comprises a thermoplastic polymer, such as thermoplastic polyurethane. For some applications, polymer 26 comprises poly(urethane urea). For some applications, polymer 26 comprises poly(urethane siloxane). For some applications, polymer 26 comprises nylon. For some applications, polymer 26 comprises an elastomer.

During the formation of fibers 40, mixture 30 can be heated until molten (e.g., within device 28). It is hypothesized that, at least for some applications, molecule 20 can confer the advantages described hereinabove, despite being present at a low concentration in mixture 30, because it may be energetically favorable for molecule 20 to migrate to the outer surface of fiber 40, and/or to assume an orientation that presents at least one fluorinated end-group 24 at the surface of the fiber, e.g., before or during solidification of the fiber.

For some applications, fibers 40 are incorporated into a yarn 50 (e.g., the yarn is formed from fibers 40), e.g., by spinning, and/or by forming a filament yarn (FIG. 1B). For some such applications, fibers 40 are formed as, or are cut into, staple fiber, and yarn 50 is formed by spinning the staple fiber into the yarn. For some applications, yarn 50 is core-spun yarn.

For some applications, yarn 50 is incorporated into a textile or fabric 60 (e.g., the textile or fabric can be formed from yarn 50), e.g., by interlacing lengths of the yarn (FIG. 1C). For example, yarn 50 can be woven or knitted into fabric/textile 60.

FIG. 2 is a flowchart that shows at least some steps of the technique described hereinabove. Arrow 32 represents formation of fiber 40 from mixture 30. Arrow 42 represents incorporation of fiber 40 into yarn 50. Arrow 52 represents incorporation of yarn 50 into fabric/textile 60.

FIG. 3 is a flowchart showing optional modifications (i.e., variants) of the technique described with reference to FIGS. 1-2, with broken lines indicating optional elements and steps.

Fabric/textile 60 can be incorporated into a medical device or implant, e.g., as described hereinbelow. For some applications in which fabric/textile 60 is produced, the fabric/textile incorporates fiber 40 by incorporating yarn 50 that itself incorporates fiber 40, e.g., as described hereinabove, and as represented by arrows 42 and 52). For some applications, the fabric/textile incorporates fiber 40 more directly, e.g., as a nonwoven fabric/textile, such as via electrospinning (e.g., solution electrospinning or melt electrospinning) or felting, and as represented by arrow 44.

There is provided, in accordance with some applications, a method comprising forming, into a fiber, a mixture that includes (a) a polymer, and (b) an additive molecule that includes one or more fluorinated end-groups; and incorporating the fiber in a yarn, fabric, textile, implant, and/or medical device. In some applications, the additive molecule includes (i) a polymeric backbone (e.g., that includes polyurethane), and (ii) fluorinated end-groups at at least one end of the backbone

Similarly, there is provided, in accordance with some applications, a fabric or textile, comprising fibers that have been formed from a mixture that comprises (1) a polymer; and (2) an additive molecule that includes one or more fluorinated end-groups. In some applications, the additive molecule includes (i) a polymeric backbone, and (ii) fluorinated end-groups at at least one end of the backbone.

For some applications, the desired product is yarn 50. For example, yarn 50 can be a thread used to stitch together components of a medical implant or medical device (e.g., as described hereinbelow), or can be a surgical suture.

There is provided, in accordance with some applications, a method comprising forming into fiber a mixture that includes (a) a polymer, and (b) an additive molecule that includes one or more fluorinated end-groups, and the method includes forming a yarn that includes the fiber.

In some applications, the additive molecule that includes (i) a polymeric backbone, and (ii) fluorinated end-groups at at least one end of the backbone.

Similarly, there is provided, in accordance with some applications, thread (e.g., surgical suture) comprising fibers formed into a yarn, the fibers having been formed from a mixture comprising (1) a polymer; and (2) an additive molecule that includes one or more fluorinated end-groups. In some applications, the additive molecule that includes (i) a polymeric backbone, and (ii) fluorinated end-groups at at least one end of the backbone.

For some applications, the desired product is fiber 40 itself, e.g., fiber 40 can be used without being incorporated into a yarn or fabric/textile, e.g., as a bulking or padding agent. For example, for some applications, a method comprises forming fibers of 5-20 micron (e.g., 10-15 micron) diameter from a mixture of (a) a polymer, and (b) an additive molecule that includes (i) a polymeric backbone, and (ii) fluorinated end-groups at at least one end of the backbone.

Although FIG. 3 shows mixture 30, arrow 32, and fiber 40 with solid lines, the scope of the disclosure also includes the possibility of receiving (e.g., acquiring) fiber 40 pre-formed, and performing only steps that are further downstream from forming the fiber.

For example, for some applications, a method can comprise forming a yarn that includes fiber formed from a mixture of (a) a polymer, and (b) an additive molecule that includes (i) a polymeric backbone, and (ii) fluorinated end-groups at at least one end of the backbone. For some applications, a method can comprise forming a fabric or textile that includes fiber formed from a mixture of (a) a polymer, and (b) an additive molecule that includes (i) a polymeric backbone, and (ii) fluorinated end-groups at at least one end of the backbone.

For some applications, yarn 50 is formed substantially from (e.g., consists substantially of) only fiber 40, and for some such applications no further substance (e.g., coating) is applied to the yarn after its formation. However, for some applications, yarn 50 is a blend of fiber 40 and another fiber 46, e.g., a fiber that does not comprise molecule 20. For example, yarn 50 can be a core-spun yarn, e.g., with fiber 46 forming a filament core having fiber 40 staple wrapped therearound.

For some applications, fabric/textile 60 is formed substantially from (e.g., consists substantially of) only fiber 40 (e.g., in the form of yarn 50), and for some such applications no further substance (e.g., coating) is applied to the fabric/textile after its formation. However, for some applications, fabric/textile 60 can be formed by interlacing lengths of yarn 50 and lengths of another yarn 54, e.g., a yarn that does not comprise molecule 20. Alternatively or additionally, fabric/textile 60 can be formed by directly incorporating into the fabric/textile another fiber 56, e.g., a fiber that does not comprise molecule 20. Therefore, for some applications, fabric/textile 60 is a blended fabric/textile.

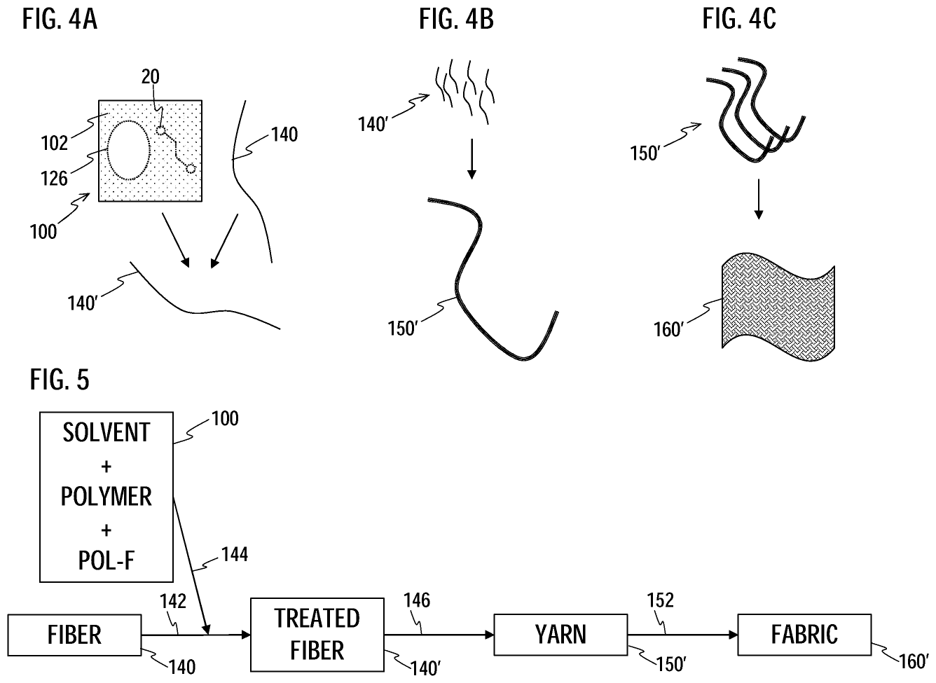

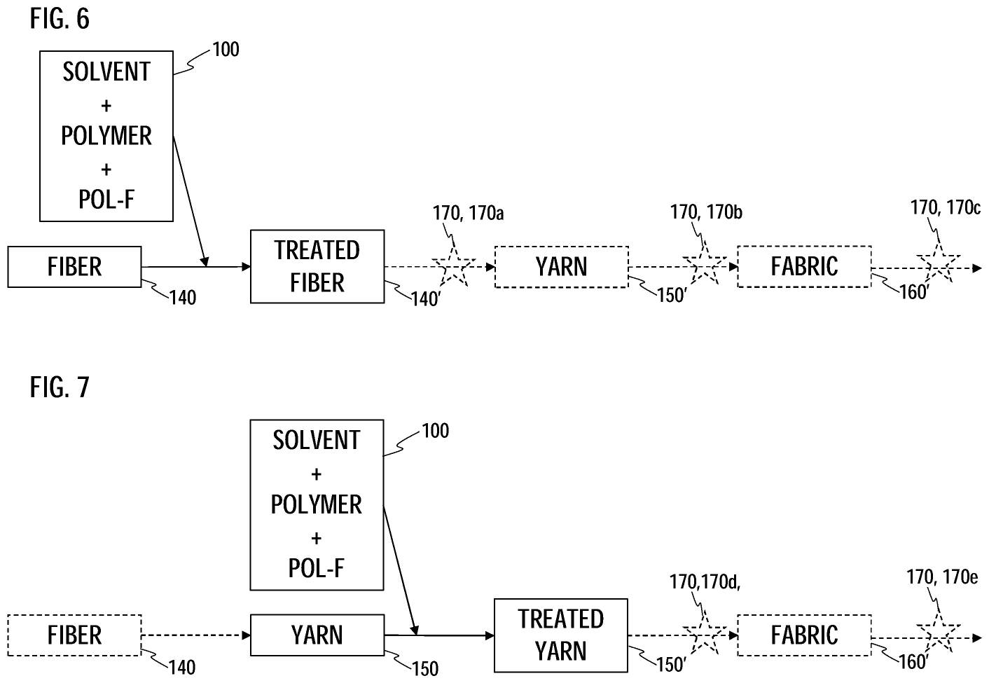

Reference is made to FIGS. 4A-C and 5-8, which are schematic illustrations, and flowcharts illustrating at least some steps, of techniques for manufacturing fiber, yarn, and/or fabric/textile, in accordance with some applications. For these techniques, the forming of the fibers, yarns, textiles, and fabrics (e.g., the structural components thereof) are as described with reference to FIGS. 1A-3, mutatis mutandis, except that the molecule 20 is not incorporated into the fiber during its production. Rather, molecule 20 is added to the surface of a fiber 140, a yarn 150, and/or a textile or fabric 160, e.g., after their formation. Other than this difference, fiber 140 generally corresponds to fiber 40, yarn 150 generally corresponds to yarn 50, and fabric/textile 160 generally corresponds to fabric/textile 60.

FIGS. 4A-C and 5 illustrate a technique in which a solution 100 is applied to fiber 140 (FIG. 4A). Solution 100 comprises molecule 20, and often a polymer 126, dissolved in a solvent 102. For some applications, polymer 126 is the same as polymer 26, described hereinabove.

For some applications, polymer 126 is different to polymer 26, e.g., monomerically, and/or with respect to molecular weight. For example, polymer 126 can have a higher or lower degree of polymerization than polymer 26. Despite this, polymer 126 can be monomerically identical to polymer 26 (e.g., having a different degree of polymerization, but with the same monomer composition/ratio). Polymer 126 can be less soluble than polymer 26 in solvent 102.

Solvent 102 is then allowed to evaporate (e.g., passively, or actively via an evaporative technique such as application of heat and/or vacuum), such that molecule 20 remains as a residue on the surface (e.g., coating the surface) of the fiber, thereby resulting in a treated fiber 140′ (FIG. 4A). In this context, the suffix ′ represents the surface presence of molecule 20. It is hypothesized that the inclusion of polymer 126 in solution 100 advantageously facilitates the concentration and/or orientation of molecule 20 at the surface.

For some applications (and in particular for some applications in which polymer 126 and/or backbone 22 comprises polyethylene terephthalate), solvent 102 comprises trifluoro acetic acid, formic acid, dichloromethane, chloroform, and/or ethylacetate. For some applications (and in particular for some applications in which polymer 126 and/or backbone 22 comprises a polyurethane), solvent 102 comprises dimethylformamide, tetrahydrofuran, dimethyl sulfoxide, and/or dioxane. For some applications (and in particular for some applications in which polymer 126 and/or backbone 22 comprises a nylon), solvent 102 comprises a phenol, a cresol, and/or dimethylformamide.

Fibers 140′ are then incorporated into a yarn, e.g., as described hereinabove, mutatis mutandis (FIG. 4B). In FIG. 4B, the yarn has been assigned reference numeral 150′ (i.e., yarn 150 has received the suffix ′) due to the surface presence of molecule 20. Subsequently, yarn 150′ is incorporated into a fabric or textile, e.g., as described hereinabove, mutatis mutandis (FIG. 4C). In FIG. 4C, the fabric/textile has been assigned reference numeral 160′ (i.e., fabric/textile 160 has received the suffix ′) due to the surface presence of molecule 20.

FIG. 5 is a flowchart that shows at least some steps of the technique described with reference to FIGS. 4A-C. Together, arrows 142 and 144 represent the treatment (e.g., coating) of fiber 140 to form treated fiber 140′. Arrow 146 represents incorporation of treated fiber 140′ into yarn 150′. Arrow 152 represents incorporation of yarn 150′ into fabric/textile 160′.

FIGS. 6-8 show optional modifications (i.e., variants) of the technique described with reference to FIGS. 4A-C and 5, in accordance with some applications. These variants are similar to the variants described with reference to FIG. 3, mutatis mutandis.

FIG. 6 indicates that, for some applications, forming fabric/textile 160′ is optional. For example, the desired product can be treated yarn 150′. For example, treated yarn 150′ can be a thread used to stitch together components of a medical implant or medical device (e.g., as described hereinbelow), or can be a surgical suture. There is provided, in accordance with some applications, a method comprising applying, to a fiber, a solution that includes (1) a solvent, (2) a polymer dissolved in the solvent, and (3) an additive molecule dissolved in the solvent, the additive molecule including one or more fluorinated end-groups. The method includes allowing the solvent to evaporate and forming a yarn (e.g., a surgical suture) that includes the fiber. In some applications, the additive molecule including (i) a polymeric backbone (e.g., that includes polyurethane), and (ii) fluorinated end-groups at at least one end of the backbone.

Similarly, there is provided, in accordance with some applications, surgical suture, comprising fibers formed into a yarn, the fibers having had a solution applied thereto, the solution comprising: a solvent, a polymer dissolved in the solvent, and an additive molecule dissolved in the solvent, the additive molecule including one or more fluorinated end-groups. In some applications, the additive molecule includes (i) a polymeric backbone, and (ii) fluorinated end-groups at at least one end of the backbone.

FIG. 6 also indicates that, for some applications, forming yarn 150′ is also optional. For example, for some applications, the desired product is treated fiber 140′ itself, e.g., treated fiber 140′ can be used without being incorporated into a yarn or fabric/textile, e.g., as a bulking or padding agent. For example, for some applications, a method comprises applying, to fibers 5-20 micron (e.g., 10-15 micron) diameter, a solution that includes (1) a solvent, (2) a polymer, dissolved in the solvent, and (3) an additive molecule, dissolved in the solvent, the additive molecule including (i) a polymeric backbone, and (ii) fluorinated end-groups at at least one end of the backbone. Such a method can further comprise allowing the solvent to evaporate.

FIG. 7 indicates that, for some applications, solution 100 is applied to yarn 150 after the yarn has been formed (rather than to fiber 140 prior to formation of the yarn), thereby resulting in treated yarn 150′. There is provided, in accordance with some applications, a method comprising applying, to a yarn, a solution that includes (1) a solvent, (2) a polymer dissolved in the solvent, and (3) an additive molecule dissolved in the solvent, the additive molecule including one or more fluorinated end-groups. The method can include allowing the solvent to evaporate. In some applications, the additive molecule including (i) a polymeric backbone, and (ii) fluorinated end-groups at at least one end of the backbone.

FIG. 7 also indicates that, for some applications, forming fabric/textile 160′ from treated yarn 150′ is optional, e.g., as described with reference to FIG. 6, mutatis mutandis. FIG. 7 further indicates that the scope of the disclosure includes the possibility of receiving (e.g., acquiring) yarn 150 pre-formed.

FIG. 8 indicates that, for some applications, solution 100 is applied to fabric/textile 160 after the fabric/textile has been formed (rather than to fiber 140 prior to formation of the yarn, or to yarn 150 prior to formation of fabric/textile 160), thereby resulting in treated fabric/textile 160′. There is provided, in accordance with some applications, a method comprising applying, to a fabric/textile, a solution that includes: (1) a solvent, (2) a polymer dissolved in the solvent, and (3) an additive molecule dissolved in the solvent, the additive molecule including one or more fluorinated end-groups; The method can include allowing the solvent to evaporate. In some applications, the additive molecule including (i) a polymeric backbone, and (ii) fluorinated end-groups at at least one end of the backbone

Similarly, there is provided, in accordance with some applications, a fabric/textile, to which a solution has been applied, the solution comprising: (1) a solvent; (2) a polymer dissolved in the solvent; and (3) an additive molecule dissolved in the solvent, the additive molecule including one or more fluorinated end-groups. In some applications, the additive molecule including (i) a polymeric backbone, and (ii) fluorinated end-groups at at least one end of the backbone.

FIG. 8 also indicates that the scope of the disclosure includes the possibility of receiving (e.g., acquiring) yarn 150 or fabric/textile 160 pre-formed.

Reference is again made to FIGS. 4A-C and 5-8. For some applications, rather than forming the residue by applying solution 100 to fiber 140, yarn 150, or fabric/textile 160, the residue can be formed using spray coating, dip coating, melt fiber extrusion, or coextrusion, e.g., using a molten mixture of polymer 126 and molecule 20, optionally without using solvent 102.

Reference is again made to FIGS. 4A-C and 5-8. As described with reference to FIGS. 1A-3, it is hypothesized that, at least for some applications, molecule 20 can confer its advantages despite being present at a low concentration in its mixture with the polymer because it may be energetically favorable for molecule 20 to migrate to the outer surface of the fiber, and/or to assume an orientation that presents at least one fluorinated end-group at the surface of the fiber, e.g., before or during solidification of the fiber. For some applications, even when molecule 20 is applied to a pre-existing fiber, yarn, or fabric/textile (e.g., as described with reference to FIGS. 4A-C and 5-8), an opportunity is provided for similar concentration and/or orientation of molecule 20 to occur by applying heat to the fiber, yarn, textile, and/or fabric after solution 100 has been applied, and generally after solvent 102 has evaporated.

That is, the application of solution 100 to fiber 140, and the subsequent evaporation of solvent 102, typically result in the formation of a residue on the fiber, the residue including polymer 126 and molecule 20. It is hypothesized that, at least for some applications, the advantageous concentration and/or orientation of molecule 20 described above might not occur during formation of the residue via evaporation of solvent 102 (or may not occur to the same extent as during solidification due to cooling). Therefore, for some applications, a heat-induced concentration step 170 is performed in which the additive molecule is concentrated toward a surface of the residue by heating the residue (e.g., the fiber covered in the residue), e.g., after the solvent has already evaporated. Subsequently, the residue (e.g., the fiber covered in the residue) is allowed to cool.

There is provided, in accordance with some applications, a method, comprising (1) applying, to a fiber, a solution that includes: a solvent, a polymer dissolved in the solvent, and an additive molecule dissolved in the solvent, the additive molecule including one or more fluorinated end-groups; (2) forming a residue on the fiber by allowing the solvent to evaporate, the residue including the polymer and the additive molecule; (3) concentrating the additive molecule toward a surface of the residue by heating the residue; and (4) subsequently, allowing the residue to cool. In some applications, the additive molecule including (i) a polymeric backbone (e.g., comprising polyurethane), and (ii) fluorinated end-groups at at least one end of the backbone.

Heat-induced concentration step 170 is represented in FIGS. 6-9 by a star. Suffixes a-f are applied to reference numeral 170 to indicate the variety of points at which step 170 can be performed.

For some applications, step 170 is performed on fiber after the residue has been formed on the fiber but before the fiber is incorporated into yarn (e.g., FIG. 6, 170a).

Similarly, for some applications, step 170 is performed on yarn after the residue has been formed on the yarn but before the yarn is incorporated into fabric/textile (e.g., FIG. 7, 170d).

For some applications, step 170 is performed on yarn (FIG. 6, 170b) or fabric/textile (FIG. 6, 170c) that incorporates fiber upon which the residue was previously formed.

For some applications, step 170 is performed on fabric/textile that incorporates yarn upon which the residue was previously formed (FIG. 7, 170e).

For some applications, step 170 is performed on fabric/textile after the residue has been formed on the fabric/textile (e.g., FIG. 8, 170f).

For some applications, step 170 is a discrete step, procedurally/or and temporally separated from the forming of the residue (e.g., from the application of the solution and drying of the solvent).

For some applications, the step of allowing solvent 102 to dry comprises heating solution 100 (e.g., the fiber, yarn, or fabric/textile upon which the solution is disposed). For some such applications, step 170 is continuous or simultaneous with the heat-drying of solvent 102. For example, heat can be applied for a longer duration than that required to merely evaporate solvent 102. Alternatively or additionally, a temperature can be used that is higher than that required to merely evaporate solvent 102.

For some applications, step 170 involves heating the residue by at least 50 degrees C. (e.g., above ambient temperature, and/or above the temperature used or required to dry solvent 102).

For some applications, polymer 126 has a different (e.g., higher or lower) melting temperature than the polymer from which fiber 140 is formed. For some applications, polymer 126 has a different (e.g., higher or lower) higher glass-transition temperature than the polymer from which fiber 140 is formed.

For some applications, step 170 involves heating the residue to a temperature that is greater than the melting temperature of polymer 126 but lower than the melting temperature of the polymer from which fiber 140 is formed.

For some applications, step 170 involves heating the residue to a temperature that is greater than the glass-transition temperature of polymer 126 but lower than the glass-transition temperature of the polymer from which fiber 140 is formed.

For some applications, step 170 involves heating the residue to a temperature that is greater than the melting temperature of polymer 126 but lower than the glass-transition temperature of the polymer from which fiber 140 is formed.

For some applications, step 170 involves heating the residue for no more than 1 minute, e.g., for no more than 30 seconds, e.g., for no more than 10 seconds, e.g., for no more than 5 seconds, such as for no more than 1 second.

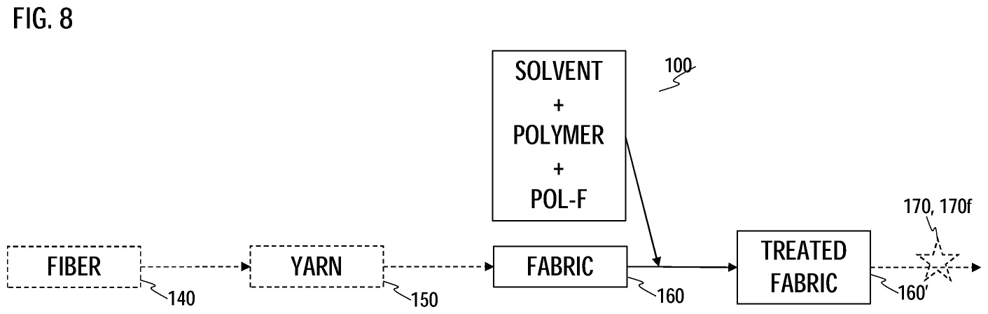

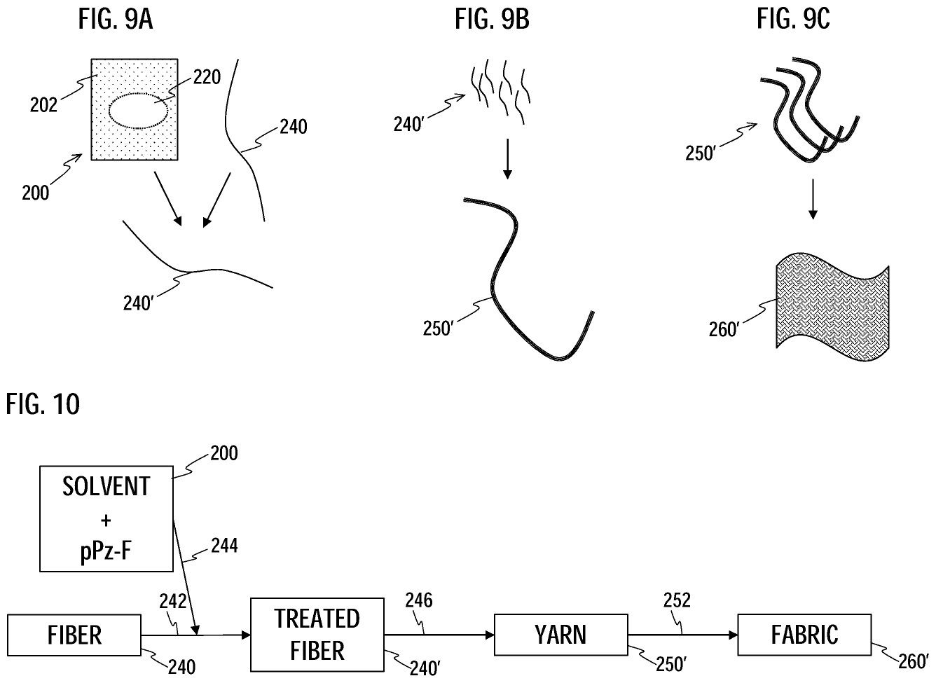

Reference is now made to FIGS. 9A-C and 10-13, which are schematic illustrations, and flowcharts illustrating at least some steps, of techniques for manufacturing fiber, yarn, and/or fabric/textile, in accordance with some applications. These techniques are typically as described with reference to FIGS. 4A-C and 5-8, mutatis mutandis. However, rather than adding molecule 20 to the surface of fiber 140, yarn 150, and/or fabric/textile 160, a molecule 220 is added.

For some applications, molecule 220 can be or comprise a surface modifying macromolecule. For some applications, molecule 220 can be or comprise a macromolecule with one or more fluorinated end-groups (end-groups are also sometimes referred to end-caps). For some applications, molecule 220 can be or comprise a fluoro-oligomer. For some applications, molecule 220 can be or comprise a low molecular weight fluoro-oligomer. For some applications, molecule 220 can be or comprise a polyphosphazene molecule. For some applications, molecule 220 can be or comprise a fluoropolymer molecule. For some applications, molecule 20 comprises a combination of two or more of the foregoing.

For some applications, molecule 220 can be or comprise a fluorinated polyphosphazene. For example, molecule 220 can be a Polyzene-F (PzF) molecule. Molecule 220 can be and is sometimes referred to herein, for illustration, as a fluorinated polyphosphazene or “pPz-F” in the figures.

Fluorinated end-groups herein can be any known or existing fluorinated end group and can be selected according to the desired chemistry for a particular application. In some applications, fluorinated end-groups can include fluoropolyether(s), perfluoropolyether(s), fluoroalcohol(s), polyfluoroalkyl(s), etc.

FIGS. 9A-C and 10 illustrate a technique in which a solution 200 is applied to a fiber 240 (FIG. 9A). Solution 200 comprises molecule 220 dissolved in a solvent 202. Solvent 202 is then allowed to evaporate (e.g., passively, or actively via an evaporative technique such as application of heat and/or vacuum), such that molecule 220 remains as a residue on the surface (e.g., coating the surface) of the fiber, thereby resulting in a treated fiber 240′ (FIG. 9A). In this context, the suffix ′ represents the surface presence of molecule 220.

Fibers 240′ are then incorporated into a yarn, e.g., as described hereinabove, mutatis mutandis (FIG. 9B). In FIG. 9B, the yarn has been assigned reference numeral 250′ (i.e., yarn 250 has received the suffix ′) due to the surface presence of molecule 220. Subsequently, yarn 250′ is incorporated into a fabric or textile, e.g., as described hereinabove, mutatis mutandis (FIG. 4C). In FIG. 9C, the fabric/textile has been assigned reference numeral 260′ (i.e., fabric/textile 260 has received the suffix ′) due to the surface presence of molecule 220.

FIG. 10 is a flowchart that shows at least some steps of the technique described with reference to FIGS. 9A-C. Together, arrows 242 and 244 represent the treatment (e.g., coating) of fiber 240 to form treated fiber 240′. Arrow 246 represents incorporation of treated fiber 240′ into yarn 250′. Arrow 252 represents incorporation of yarn 250′ into fabric/textile 260′.

It is hypothesized that the presence of molecule 220 on the surface of fibers 240 confers advantageous properties on the fibers (and on yarns and fabrics/textiles in which the fibers are incorporated) compared to similar fibers that are not coated with molecule 220. For example, it is hypothesized that, for applications in which fibers 240 are used within the human body (e.g., as described in more detail hereinbelow), the presence of molecule 220 advantageously inhibits thrombogenesis, fouling, and bacterial adherence and growth, and/or encourages endothelialization (attraction, binding, and/or growth of endothelial cells) on the surface of the fibers. For example, molecule 220 can be configured to promote a thin layer of endothelium to promote formation of a smooth benign surface (e.g., on a fabric, textile, medical implant, medical device, etc.) for native anatomy to interact with, while preventing over thickening (e.g., preventing the fabric, textile, medical implant, medical device, etc. from becoming too thick with endothelium and losing desired flexibility).

FIGS. 11-13 show optional modifications (i.e., variants) of the technique described with reference to FIGS. 9A-C and 10, in accordance with some applications. These variants are similar to the variants described with reference to FIGS. 6-8, mutatis mutandis.

FIG. 11 indicates that, for some applications, forming fabric/textile 260′ is optional. For example, the desired product can be treated yarn 250′. For example, treated yarn 250′ can be a thread used to stitch together components of a medical implant or medical device (e.g., as described hereinbelow), or can be a surgical suture.

There is provided, in accordance with some applications, a method comprising applying, to a fiber, a solution that includes (1) a solvent, and (2) a fluorinated polyphosphazene. The method can further include allowing the solvent to evaporate and forming a yarn that includes the fiber.

Similarly, there is provided, in accordance with some applications, surgical suture, comprising fibers formed into a yarn, the fibers having had a solution applied thereto, the solution comprising: a solvent and a fluorinated polyphosphazene.

FIG. 11 also indicates that, for some applications, forming yarn 250′ is also optional. For example, for some applications, the desired product is treated fiber 240′ itself, e.g., treated fiber 240′ can be used without being incorporated into a yarn or fabric/textile, e.g., as a bulking or padding agent. For example, for some applications, a method comprises applying, to fibers of 5-20 micron (e.g., 10-15 micron) diameter, a solution that includes (1) a solvent, and (2) a fluorinated polyphosphazene. Such a method can further comprise allowing the solvent to evaporate.

FIG. 12 indicates that, for some applications, solution 200 is applied to yarn 250 after the yarn has been formed (rather than to fiber 240 prior to formation of the yarn), thereby resulting in treated yarn 250′. There is provided, in accordance with some applications, a method comprising: applying, to a yarn, a solution that includes (1) a solvent, and (2) a fluorinated polyphosphazene; and allowing the solvent to evaporate.

FIG. 12 also indicates that, for some applications, forming fabric/textile 260′ from treated yarn 250′ is optional, e.g., as described with reference to FIG. 11, mutatis mutandis. FIG. 12 further indicates that the scope of the disclosure includes the possibility of receiving (e.g., acquiring) yarn 250 pre-formed.

FIG. 13 indicates that, for some applications, solution 200 is applied to fabric/textile 260 after the fabric/textile has been formed (rather than to fiber 240 prior to formation of the yarn, or to yarn 250 prior to formation of fabric/textile 260), thereby resulting in treated fabric/textile 260′. There is provided, in accordance with some applications, a method comprising: applying, to a fabric/textile, a solution that includes: (1) a solvent, and (2) a fluorinated polyphosphazene dissolved in the solvent; and allowing the solvent to evaporate.

Similarly, there is provided, in accordance with some applications, a fabric/textile, to which a solution has been applied, the solution comprising: (1) a solvent; and (2) a fluorinated polyphosphazene.

FIG. 13 also indicates that the scope of the disclosure includes the possibility of receiving (e.g., acquiring) yarn 250 or fabric/textile 260 pre-formed.

Reference is again made to FIGS. 4A-13, which relate to application solutions to fibers, yarns, and/or fabrics/textiles. For some applications, the application of the solution is performed by spraying. For some applications, the application of the solution is performed by dipping. For some applications, fibers 140 and/or 240 are synthetic fibers, e.g., comprising a polyester (e.g., a polyethylene terephthalate homopolymer or copolymer), a thermoplastic polyurethane, nylon, and/or an elastomer. For some applications, fibers 140 and/or 240 are natural fibers. For some applications, fibers 140 are cellulosic fibers, e.g., comprising cotton or a modified cellulose such as carboxymethyl cellulose.

For some applications, solution 100 and/or solution 200 are prepared just prior to their application, by dissolving the solute(s) in the solvent. For some applications, solution 100 and/or solution 200 are received (e.g., acquired) pre-prepared.

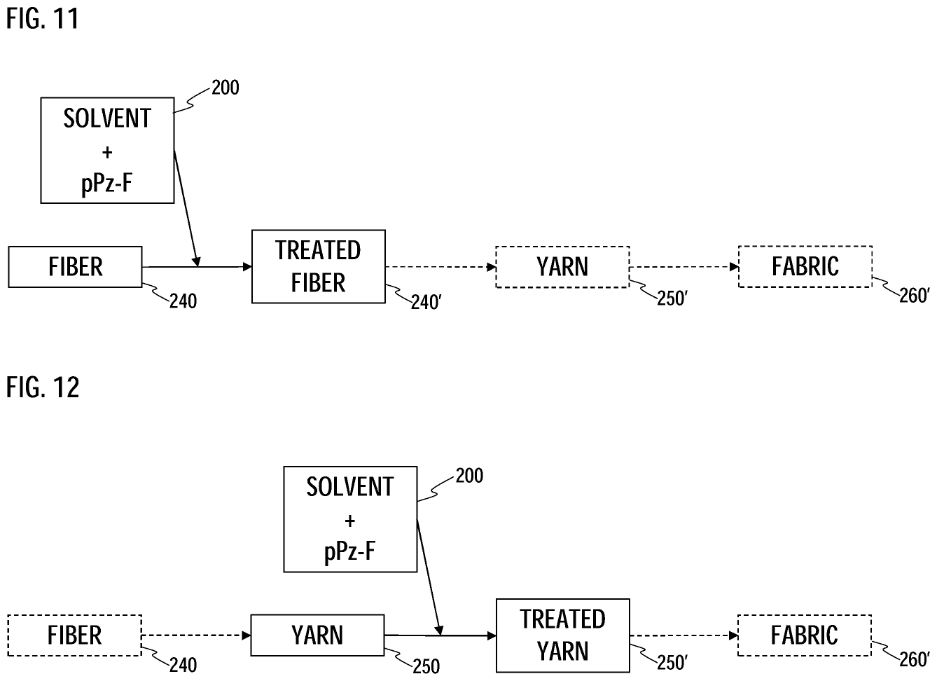

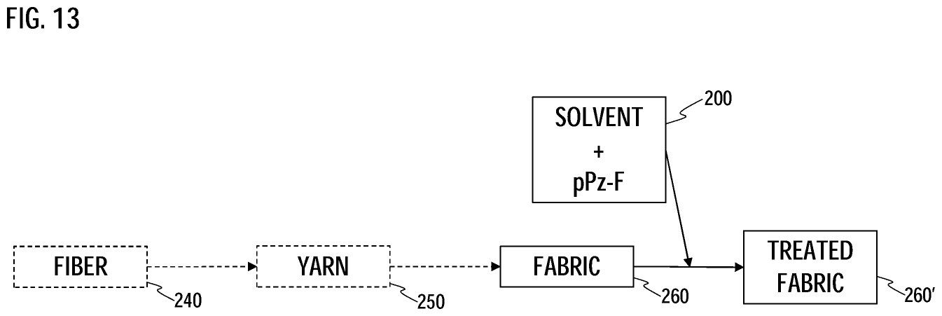

Reference is now made to FIGS. 14, 15, and 16, which are schematic illustrations of example medical implants or medical devices that comprise one or more of the fibers, yarns, textiles, and/or fabrics described hereinabove. A variety of other medical implants and/or medical devices, even though not expressly described or shown, can also include one or more of the fibers, yarns, textiles, and/or fabrics herein. The examples shown are cardiac implants or devices, for use with a heart of a subject. As described elsewhere herein, it is hypothesized that the use of molecule 20 and/or molecule 220 can confer advantages on such medical implants or devices, such as reduced thrombogenesis, fouling, and bacterial adherence and growth, and/or enhanced endothelialization. Examples of such implants or devices are shown herein as a prosthetic valve 300 (FIG. 14), an annuloplasty structure 320 (FIG. 15), and a coaptation-assistance implant 340. In some applications, molecule 20 and/or molecule 220 can be configured to promote a thin layer of endothelium to promote formation of a smooth benign surface on a fabric, textile, medical implant, medical device, etc. for native anatomy to interact with (e.g., the thin endothelium may interact with the body as native tissue). Molecules 20 and 220 are configured to preventing over thickening (e.g., preventing the fabric, textile, medical implant, medical device, etc. from becoming too thick with endothelium) and losing desired flexibility.

The example prosthetic valve 300 shown in FIG. 14 comprises a frame 302 (e.g., a metallic frame, a stent frame, etc.), and prosthetic leaflets 304 supported by the frame. Frame 302 can be dressed (e.g., partly or completely covered and/or lined) by a textile or fabric 306 (e.g., a cover or covering comprising the textile/fabric 306), e.g., to facilitate and/or direct blood flow through the prosthetic valve and/or to inhibit paravalvular leakage. Other types of covers or coverings comprising the textile/fabric 306 can also be used and can be positioned in a variety of ways inside, outside, and/or around the frame. Other types of frames than that shown are also possible. Leaflets 304 and/or fabric/textile 306 can be coupled to frame 302 by stitches of a thread (e.g., a surgical suture) 308. Fabric/textile 306 and/or thread 308 can include and/or be coated with molecule 20 and/or molecule 220, as described hereinabove.

There is provided, in accordance with some applications, a medical implant or device that comprises fibers to which a solution has been applied, the solution comprising (1) a solvent; (2) a polymer, dissolved in the solvent; and (3) an additive molecule, dissolved in the solvent, the additive molecule including (i) a polymeric backbone, and (ii) fluorinated end-groups at at least one end of the backbone. There is provided, in accordance with some applications, a medical implant or device that comprises fibers to which a solution has been applied, the solution comprising (1) a solvent; (2) a fluorinated polyphosphazene.

For some applications, frame 302 itself can be coated with molecule 20 and/or molecule 220, e.g., as described hereinabove, mutatis mutandis. Furthermore, for some applications, leaflets 304 can have had molecule 20 and/or molecule 220 applied thereto, e.g., as described hereinabove, mutatis mutandis.

The example annuloplasty structure 320 shown in FIG. 15 comprises a sleeve 322 (e.g., a tubular structure) formed from a textile or fabric that can include and/or be coated with molecule 20 and/or molecule 220, as described hereinabove. Annuloplasty structure 320 can further comprise a plurality of anchors 324, a flexible elongated contracting member (e.g., a wire, suture, line, braid, thread, chain, etc.) 326, and/or an adjustment mechanism 328 (e.g., a spool, winch, gear(s), motor, tensioner, manipulator, etc.). For some applications, annuloplasty structure 320 and/or features thereof are, mutatis mutandis, as described in U.S. Pat. No. 9,949,828 to Sheps et al. and/or U.S. Pat. No. 10,765,514 to Iflah et al., each of which is incorporated herein by reference in its entirety for all purposes.

For some applications, metallic components of annuloplasty structure 320, such as anchors 324, contracting member 326, and/or adjustment mechanism 328, can be coated with molecule 20 and/or molecule 220, e.g., as described hereinabove, mutatis mutandis. For some applications, annuloplasty structure 320 does not comprise a sleeve. For some applications, annuloplasty structure 320 comprises a series of anchors and a contracting member (e.g., a wire, line, suture, braid, thread, chain, etc.) extending through eyelets of the anchors without the use of a sleeve. For some applications, annuloplasty structure 320 does not comprise an adjustment mechanism. For some applications, annuloplasty structure 320 and/or features thereof are, mutatis mutandis, as described in International Patent Application (PCT) publication WO 2021/084407 to Kasher et al., US Patent Application Publication 2021/0145584 to Kasher et al., and/or International Patent Application PCT/IB2022/051099 to Shafigh et al., filed Feb. 8, 2022, each of which is incorporated herein by reference in its entirety for all purposes.

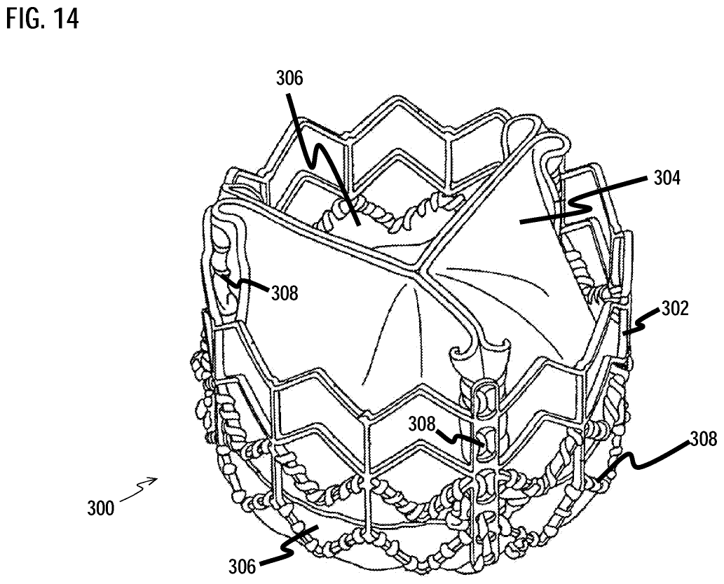

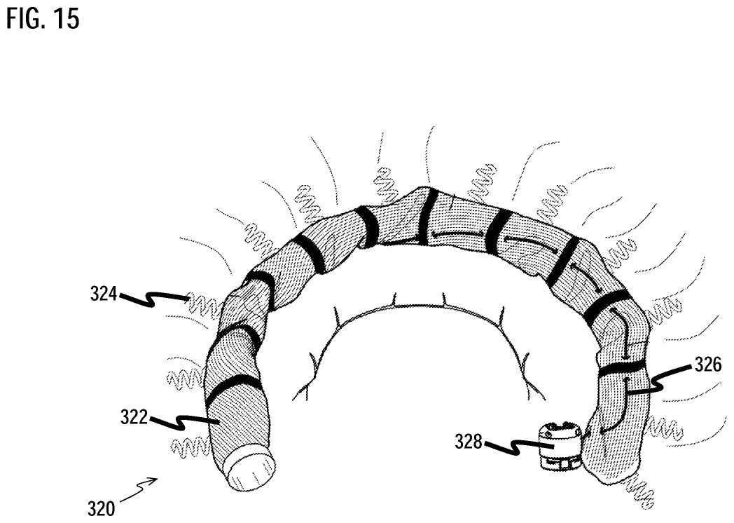

The example coaptation-assistance implant 340 shown in FIG. 16 comprises an interface 342, and a flexible coaptation portion 344 coupled to the interface. The coaptation portion can be configured in a variety of ways, for example, as a leaf, sheet, mesh, wire form, membrane, spacer, expandable member, inflatable member, balloon, foam, combination of two or more of these, etc. The interface 342 can also be configured in a variety of ways, for example, as an anchor mount, multiple anchor mounts, one or more anchor portions, one or more hubs, one or more connections, one or more sockets, etc. The interface can be configured to be attached, secured, or otherwise anchored to tissue with one or multiple anchors, fixation elements, etc.

In some applications, coaptation portion or leaf 344 comprises (i) a flexible wire or wire form 346, shaped as a loop that defines an aperture 347 therethrough (other shapes of wires or wire forms are also possible, e.g., multiple loops, struts, apertures, shapes, sizes, etc.), and (ii) a textile or fabric 348 (or cover/covering comprising the textile/fabric 348), coupled to the wire and covering at least part of the aperture. That is, wire 346 serves as a frame, and is dressed with fabric/textile 348 or a cover/covering comprising the fabric/textile 348. Implant 340 can further comprise an anchor 350 (a head of which is visible in FIG. 16).

In some applications, implant 340 is implanted using a delivery system that comprises a shaft and an anchor driver (not shown). The shaft can be configured to, via engagement with the interface, position the implant in a position in which the interface is at a site in the heart of a subject proximate a valve of the heart, and the coaptation portion extends over a first leaflet of the valve and toward a second leaflet of the valve. For example, in the position, the interface can be on the posterior annulus of the mitral valve of the heart, and the leaf can extend over the posterior leaflet of the mitral valve and toward the anterior leaflet of the mitral valve. The anchor driver can be configured to secure the implant in the position by anchoring the interface to tissue of the heart by driving anchor 350 into the tissue (e.g., at the site in the heart). Fabric/textile 348 can include and/or be coated with molecule 20 and/or molecule 220, as described hereinabove. For some applications, implant 340 and/or features thereof are, mutatis mutandis, as described in U.S. Provisional Patent application 63/124,704 to Chau et al., filed Dec. 11, 2020, and entitled “Systems and methods for heart valve leaflet repair,” and/or International Patent Application (PCT) Publication WO 2022/006087 to Chau et al., each of which is incorporated herein by reference in its entirety for all purposes.

For some applications, and as shown, wire 346 is arranged to define the loop as teardrop-shaped.

For some applications, and as shown, coaptation portion 344 has an open part 352 at which aperture 347 is not covered by the fabric/textile or a cover/covering comprising the fabric/textile (e.g., is not covered by any fabric/textile).

For some applications, and as shown, coaptation portion 344 has a root 354 that is coupled to interface 342, and a tip 356 at an opposite end of the coaptation portion from the root, and fabric/textile 348 is disposed between open part 352 and tip 356.

For some applications, metallic components of implant 340, such as anchor 350 and/or wire 346, can be coated with molecule 20 and/or molecule 220, e.g., as described hereinabove, mutatis mutandis.

Reference is again made to FIGS. 14, 15, and 16. For some applications, a given implant can comprise molecule 20 and molecule 220. As one example, and as shown, implant 340 can comprise a first fabric/textile 348a that includes and/or is coated with one of molecule 20 and molecule 220, and a second fabric/textile 348b that includes and/or is coated with the other of molecule 20 and molecule 220. The molecule 20 and fabric/textile 348a can comprise fluorinated end-groups with different chemical structures from the fluorinated end-groups that molecule 220 and fabric/textile 348b comprise.

In some applications, fabric/textile 348b can be disposed proximate to anchor 350 and can include and/or be coated with molecule 220 in order to enhance endothelialization, e.g., to augment anchoring to the tissue at the anchoring site (e.g., the annulus of the valve). In some applications, fabric/textile 348a can be disposed closer to lower half of the implant or closer to tip 356 and can be positioned to contact a leaflet (e.g., an opposing leaflet) of the valve, and can include and/or be coated with molecule 20 in order to inhibits thrombogenesis, fouling, and bacterial adherence and growth. In some applications, fabric/textile 348a can be disposed closer to lower half of the implant or closer to tip 356 and can be positioned to contact a leaflet (e.g., an opposing leaflet) of the valve, and can include and/or be coated with molecule 20 in order to promote a thin layer of endothelium to promote a smooth benign surface for the native valve to coapt against. Where only a thin layer of endothelium is produced, the implant can maintain needed flexibility without over thickening.

In some applications, fabric/textile 348a can be disposed closer to lower half of the implant or closer to tip 356 and can be positioned to contact a leaflet (e.g., an opposing leaflet) of the valve, and can include and/or be coated with silicone to inhibit thrombogenesis, fouling, and bacterial adherence and growth (and may or may not include molecule 20 or molecule 220).

In some applications, additional fabric/textiles can also be used, for example, a third fabric/textile and/or a fourth fabric textile (or more) that includes and/or is coated with another version of molecule 20 and molecule 220, each with different fluorinated end-groups for different surface properties.

With a variety of fabrics/textiles and/or a variety of coating herein (each with different fluorinated end-groups) positioned or used at different locations on a medical implant or medical device, different desired surface chemistries can be obtained in different locations on the implant or device. This can allow for optimizing the medical implant or device for a particular part of the body and for a particular function.

For some applications, a given implant or device (e.g., a frame thereof) can be dressed with a nonwoven fabric/textile, e.g., by spinning fiber directly onto a frame of the implant or device (e.g., using solution electrospinning, melt electrospinning, jet spinning, etc.). For some such applications, the fiber can include molecule 20 or be integrally formed therewith (e.g., the fiber can be fiber 40). In some applications, the fiber can be coated with molecule 20 or molecule 220 after being electrospun onto the frame. For some applications, different parts of the implant or device (e.g., the frame thereof) can be dressed/covered (e.g., electrospin-dressed) with different fibers that include and/or are coated with different molecules.

Reference is again made to FIGS. 1A-16. For some applications, a given fiber can include and/or be coated with both molecule 20 and molecule 220. For example, a given fiber can (i) include molecule 20 or be integrally formed with molecule 20 as described with reference to FIG. 1A, and (ii) be coated with molecule 220 as described with reference to FIG. 4A.

For some applications, a given yarn can comprise both (i) fibers that includes and/or are coated with molecule 20, and (ii) fibers that are coated with molecule 220.

For some applications, a given fabric/textile can comprise both (i) yarn that includes and/or is coated with molecule 20, and (ii) yarn that is coated with molecule 220.

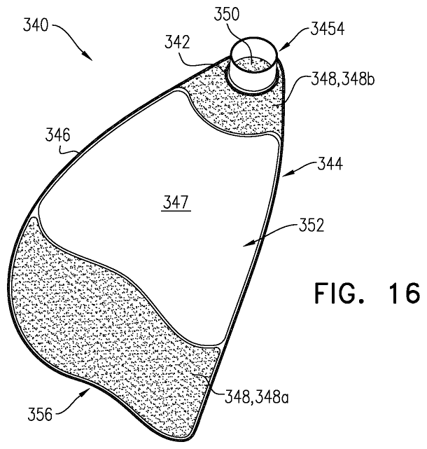

Reference is now made to FIG. 17, which is a schematic illustration illustrating at least some steps of a technique for manufacturing a stabilized fabric in accordance with some applications. The stabilized fabric is formed of a yarn, which includes an implantable yarn 402 or fiber at least partially coated by a thermoplastic material 404. Yarn 402 can, for example, be a polyester yarn, e.g., comprising a polyethylene terephthalate (such as a polyethylene terephthalate homopolymer or copolymer). Thermoplastic material 404 can, for example, be, or include, TPU (thermoplastic polyurethane), FEP (fluorinated ethylene propylene), UHMWPE (ultrahigh molecular weight polyethylene), and/or ePTFE (expanded polytetrafluoroethylene). In a first step of the technique, the yarn is formed using any suitable method or technique. Some exemplary techniques for forming the yarn are shown at sections A, B, and C of FIG. 17, and are described herein.

In the example shown in section A, implantable yarn 402a is coated with thermoplastic material 404a, to form a fully coated yarn 400a. The coating process can be carried out, for example, by applying thermoplastic 404a onto yarn 402a, as shown at reference numeral 406. This application of thermoplastic 404a is schematically illustrated at reference numeral 406 as spray-coating, but the scope of the present disclosure includes other coating techniques such as, but not limited to, dip-coating. As another example, the thermoplastic 404a can be co-extruded with yarn 402a, as shown at reference numeral 408. For some applications, implantable fibers included in yarn 402a can be coated with thermoplastic 404a, e.g., by spraying or by co-extrusion, prior to twisting or otherwise forming yarn 402a, such that when yarn 402a is formed, it is already, in effect, coated with thermoplastic 404a. In the example shown in section B, a core of implantable yarn 402b has a fiber of thermoplastic 404b spun therearound, e.g., forming a partially coated yarn 400b. In the example shown in section C, an implantable yarn 402c is co-twisted with a thermoplastic yarn 404c, e.g., to form a partially coated yarn 400c.

Following formation of the coated yarn, strands of the coated yarn 400 (e.g., 400a, 400b, or 400c), are formed into a fabric 411, e.g., by weaving or knitting. In some applications, such as that shown in FIG. 17, fabric 411 is a mesh fabric (e.g., open weave or open knit), defining gaps between weft strands 412 as well as gaps between the warp strands 414, such that windows 418 are formed in the fabric. In some applications, such as that shown in FIG. 17, fabric 411 is a leno fabric, having two warp strands 414a and 414b woven around weft strands 412. In some applications, weft strands 412 comprise coated yarn 400 (e.g., yarn 400a, yarn 400b, or yarn 400c). In some applications, warp strands 414 comprise coated yarn 400. For applications in which fabric 411 is a leno fabric, one or both of warp strands 414 (e.g., warp strands 414a and/or warp strands 414b) comprise coated yarn 400. In some applications, the yarns of fabric 411 that comprise coated yarn 400 may all be of a single type, e.g., all strands of yarn 400a, or all strands of yarn 400b. In other applications, fabric 411 can combine strands of different types, e.g., can include some strands of yarn 400a and other strands of yarn 400b.

Subsequently, junction points 416 between the warp and weft strands of fabric 411 are heat set, to fix the thermoplastic coatings of the warp and weft strands to one another, thereby to reinforce the fabric 411. For example, in some applications, the thermoplastic coating is heat set by heat pressing the fabric, for example between two heated plates or two heated rollers. In some applications, for example when smaller sections of fabric are formed, each junction point 416 can be individually heated, for example using a local heating tool similar to a soldering iron.

For some applications, following heat-setting of the fabric, windows 418 have substantially fixed dimensions. In some applications, a length of windows 418, indicated by L in FIG. 17, is not greater than 2 mm, e.g., not greater than 1.8 mm, e.g., not greater than 1.5 mm, e.g., greater than 1.2 mm, such as not greater than 1 mm. In some applications, the length L is not smaller than 0.5 mm, e.g., not smaller than 0.8 mm, such as not smaller than 1 mm. In some applications, a width of windows 418, indicated by W in FIG. 17, is not greater than 2 mm, e.g., not greater than 1.8 mm, e.g., not greater than 1.5 mm, e.g., not greater than 1.2 mm, such as not greater than 1 mm. In some applications, the width W is not smaller than 0.5 mm, e.g., not smaller than 0.8 mm, such as not smaller than 1 mm.

For some applications, fabric 411 has a thickness not greater than 80 μm, e.g., not greater than 75 μm, e.g., not greater than 70 μm, such as not greater than 65 μm. Such thicknesses of the fabric may be desirable for some applications, such as some implantable devices, e.g., artificial cardiac valves.

Advantageously, fabric 411 may be more mechanically stable than comparable fabrics that are not heat set. For example, the threads of fabric 411 may be less prone to shifting with respect to each other than threads in a comparable fabric that is not heat set. This may be particularly advantageous for applications in which fabric 411 is to be stitched, for example to a second piece of fabric 411, to another fabric, or to another structure such as a frame of a medical implant such as a prosthetic heart valve. Due to the heat setting of junctions 416, the stitches stitched through fabric 411 are less likely to cause shifting of the threads of the fabric and/or to pull the fabric apart or cause it to disintegrate.

Fabric 411 and/or the techniques for manufacture thereof may be particularly advantageous for applications in which windows 418 are desired (e.g., when an open weave is desired), such as for applications in which permeability of the fabric is desired. In contrast, other techniques for stabilizing an open-weave fabric can include applying a substance to the already-woven (or already-knit) fabric in a manner that does, or may, seal up at least some of the windows in the fabric. Examples of such other techniques include laminating, dipping, and spraying substances that adhere to the threads of the fabric.

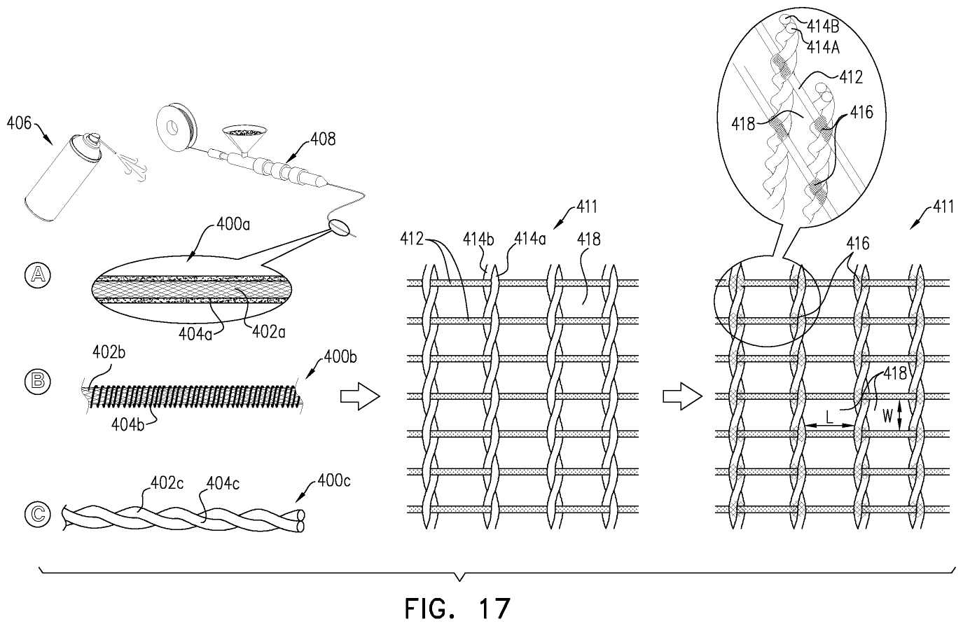

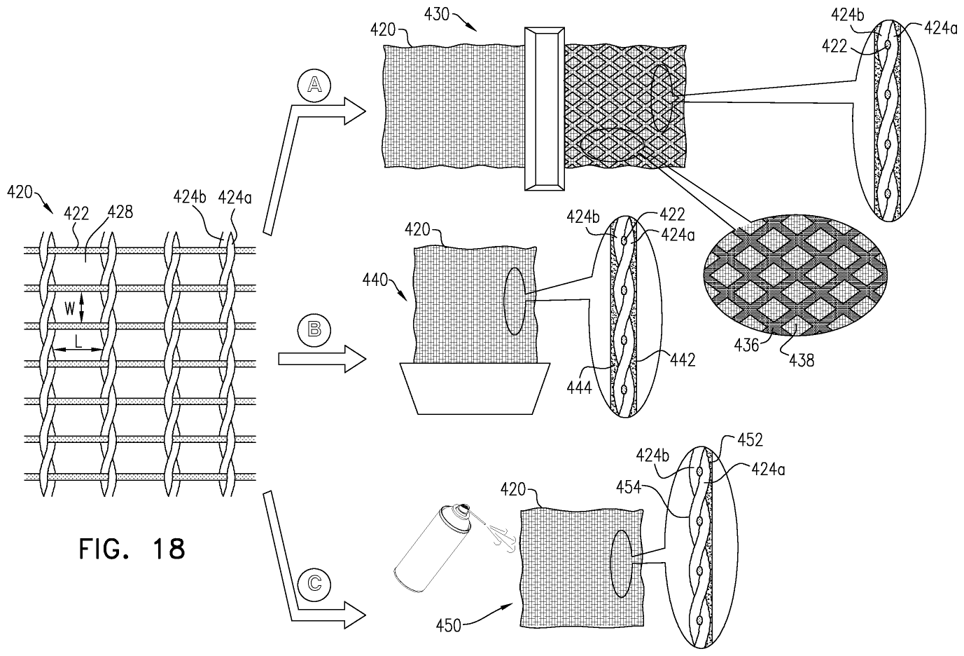

For some applications, such as for applications in which windows 418 are not desired, or are undesirable, following heat-setting of junctions 416, fabric 411 can be laminated, e.g., using one or more of the techniques described hereinbelow with respect to FIG. 18, mutatis mutandis. For some such applications, only one side of fabric 411 is laminated. For other applications, both sides of fabric 411 are laminated.

For some applications in which fabric 411 is to be a component of a medical device (e.g., an implant), the yarns of fabric 411 are adapted to prevent, or limit, tissue growth thereon. For some applications, one or more of the yarns can be adapted to encourage tissue growth thereon. For some applications, fabric 411 can be used as a skirt of an implantable device, such as an artificial cardiac valve.

Reference is now made to FIG. 18, which is a schematic illustration illustrating at least some steps of a technique for manufacturing a coated fabric in accordance with some applications. The coated fabric is formed of implantable yarn, and is at least partially coated with a thermoplastic polymer. The following description relates to a woven fabric. However, it is to be appreciated that the technique described herein is equally germane to a loosely knit fabric, and its application to a loosely knit fabric is considered within the scope of the present application.

Strands of an implantable yarn, such as a PET (polyethylene terephthalate) yarn, are formed into a mesh fabric, such as a loosely woven fabric 420 or a loosely knit fabric. In some applications, such as that shown in FIG. 18, the mesh fabric is woven, and defines gaps between weft strands 422 as well as gaps between the warp strands, such that windows 428 are formed in the fabric. In some applications, such as that shown in FIG. 18, the woven fabric is a leno fabric, having two warp strands 424a and 424b woven around the weft strands.

Following formation of fabric 420, at least one side of the fabric is at least partially coated with a polymer (e.g., a thermoplastic polymer). For some applications, the polymer can include TPU (thermoplastic polyurethane), FEP (Fluorinated ethylene propylene), UHMWPE (ultrahigh molecular weight polyethylene), and/or ePTFE (expanded polytetrafluoroethylene). The polymer may stabilize the fabric, e.g., inhibiting movement between warp and weft strands.

As shown at option A, for some applications, fabric 420 is coated by lamination thereof, as indicated at reference numeral 430. For some applications, the fabric 420 is laminated from both sides thereof, resulting in a first laminated surface 432, and a second laminated surface 434, having fabric 420 disposed therebetween. For some applications, only one side of fabric 420 is laminated.

For applications in which it is desired that fabric 420 be permeable (e.g., open weave), the lamination may be only partial, defining laminated regions 436 and non-laminated, or open, regions 438. This may advantageously combine benefits of open-weave fabric (e.g., permeability) with those of laminated fabrics (stability). For some applications, open regions 438 may be differently sized to windows 428. For example, open regions 438 can be wider than windows 428 (e.g., more than twice as wide), and/or longer than the windows (e.g., more than twice as long). Similarly, each open region 438 can cover an area at least 4 times greater (e.g., at least 8 times greater) than each window 428. Thus, multiple (e.g., more than 5, such as more than 10) entire windows 428 can appear within each non-laminated region 438.

For some applications, and as shown, the laminated regions 436 are lines that inter-cross. These lines can be 2-4 (e.g., 3) mm wide. These lines may not be parallel to the yarns of fabric 420, such as, not disposed along the weft and warp yarn. Furthermore, and as shown, these lines may not be at 45 degrees to the weft and warp yarns, but rather may be aligned at less than 45 degrees (e.g., no more than 35 degrees) and/or at least 25 degrees with respect to the weft strands. For some applications, laminated regions 436 form a lattice, defining rhombus-shaped open regions 438 therebetween. For some such applications, such rhombus-shaped open regions have a corner-to-corner length of 6-8 (e.g., 7) mm and a corner-to-corner width of 4-6 (e.g., 5) mm. For some applications, when fabric 420 is laminated, the corner-to-corner length of rhombus-shaped open regions 438 may be substantially parallel with weft strands 422 of the fabric, while the corner-to-corner width of the rhombus-shaped open regions may be substantially parallel with warp strands 424 of the fabric.

For some applications, such partial lamination can be achieved by laminating lines in one direction and separately laminating lines in another direction, e.g., so that the lines overlap in a lattice pattern. For some applications, a film of laminate with pre-formed holes in it can be used, the holes becoming unlaminated regions 438.

As shown at option B, for some applications, fabric 420 is coated by dipping it in a coating solution, as indicated at reference numeral 440. For some such applications, the fabric 420 is coated on both sides, resulting in a first coated surface 442 and a second coated surface 444, having fabric 420 disposed therebetween. As shown at option C, for some applications, fabric 420 is coated by spraying a coating solution thereon, as indicated at reference numeral 450. For some such applications, and as shown, the fabric 420 can be coated on only one side thereof, resulting in a coated surface 452, and an exposed fabric surface 454. For some applications, spray-coating can be performed on both sides.

For applications which use dip coating (option B) or spray coating (option C), THF (tetrahydrofuran), DMAc (dimethylacetamide), or acetone can be used as a solvent. The flow and pressure used for the spraying, or the amount of time the fabric is dipped into the solution, are selected to ensure a desired thickness of the resulting coated fabric, as detailed hereinbelow. Furthermore, this can determine whether windows 428 become sealed up by the coating material or remain open. Following dipping or spraying of the fabric with the coating solution, solvents can be allowed to evaporate and the coated fabric can be washed, dried, and heat pressed to obtain a uniform structure and to assist in controlling the thickness of the resulting fabric.

For some applications, following coating of the fabric, windows 428 can have substantially fixed dimensions. In some applications, a length of windows 428, indicated by L in FIG. 18, is not greater than 2 mm, not greater than 1.8 mm, not greater than 1.5 mm, not greater than 1.2 mm, or not greater than 1 mm. In some applications, the length L is not smaller than 0.5 mm, not smaller than 0.8 mm, or not smaller than 1 mm. In some applications, a width of windows 428, indicated by W in FIG. 18, is not greater than 2 mm, not greater than 1.8 mm, not greater than 1.5 mm, not greater than 1.2 mm, or not greater than 1 mm. In some applications, the width W is not smaller than 0.5 mm, not smaller than 0.8 mm, or not smaller than 1 mm. For some applications, heat and/or pressure applied to fabric 420 during coating thereof ensure that the structure of windows 428 is maintained, without delamination and/or wear of the fabric.

For some applications, fabric 420 has a thickness not greater than 80 μm, not greater than 75 μm, not greater than 70 μm, or not greater than 65 μm. Such thicknesses of the fabric may be desirable for some applications, such as some implantable device, e.g., artificial cardiac valves.

For some applications, the coating of the fabric includes an intermediate layer, which can be a thermoplastic adhesive polymer such as TPU, acrylate, siloxane, or silicone. The intermediate layer can strengthen the interaction between the fabric and coating layer, thereby limiting wear and tear of the fabric.

For some applications, e.g., when used in a medical device, the coated fabric can be scoured and sterilized, prior to use thereof.

For some applications, e.g., when used within a medical device, fabric 420 is adapted to prevent, or limit, tissue ingrowth into windows 428. For some applications, fabric 420 can be used as a skirt of an implantable device, such as an artificial cardiac valve.

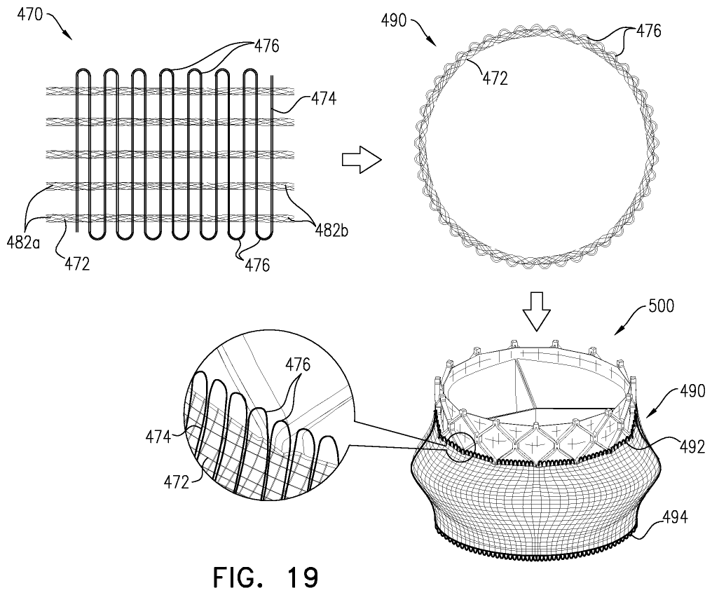

Reference is now made to FIG. 19, which is a schematic illustration illustrating at least some steps of a technique for manufacturing a fabric 470, and for using the manufactured fabric in an implantable device, in accordance with some applications.

As seen in FIG. 19, fabric 470 is woven from multiple warp strands 472 and a single weft strand 474 which goes back and forth between the warp strands, forming turns 476 at lateral edges of the fabric. Warp strands 472 can be formed of a multifilament yarn, such as a twisted or textured PET yarn. Weft strand 474 is a monofilament yarn. For some applications, monofilament weft strand 474 comprises a polymer such as PET. For some applications, monofilament weft strand 474 comprises a metal such as nitinol (e.g., annealed nitinol), cobalt-chrome, or stainless steel, e.g., the monofilament weft strand can be a metallic wire. For some applications, fabric 470 can include an additional weft strand of a twisted or textured multifilament yarn (not shown).

For some applications, following weaving of fabric 470, the fabric can be heat-pressed, for example as described hereinabove with respect to FIG. 17 or FIG. 18, mutatis mutandis. Heat-pressing can be carried out at a temperature of 180 degrees C. for a duration of 10 minutes.

For some applications, following weaving of fabric 470, the fabric can be laminated from one or both sides thereof, for example as described hereinabove with respect to FIG. 18. For example, the lamination can be carried out using aliphatic TPU, at 150° C., for a duration of three minutes. The lamination can be partial lamination having specifically shaped laminated regions and non-laminated regions, as described hereinabove with respect to FIG. 18.

For some applications, fabric 470 has a thickness not greater than 80 μm, not greater than 75 μm, not greater than 70 μm, or not greater than 65 μm.

Due to the use of monofilament yarn as the weft strands of fabric 470, the fabric is stiffer and/or more resilient in the direction of the weft strands, than in the direction of the warp strands. As such, when used, the fabric can be oriented such that the weft strands are aligned with an axis along which the fabric is desired to be stiffer and/or more resilient.

In the example shown in FIG. 19, fabric 470 is used to form a circumferential pouch 490 circumscribing a tubular implant such as a prosthetic heart valve 500. To form pouch 490, ends 482a and 482b of warp strands 472 can be brought together to form a ring that is mounted on valve 500 (e.g., on a frame of the valve) with warp strands 472 extending circumferentially, and weft strand 474 extending back and forth longitudinally, i.e., substantially perpendicularly to the circumference of the valve and/or along the height of the ring). Thus, the lateral edges of fabric 470 typically define an upper (e.g., upstream) rim 492 and a lower (e.g., downstream) rim 494 of the ring (e.g., of pouch 490). Turns 476 can therefore be disposed at rims 492 and 494.

Pouch 490 is configured to bulge away from valve 500 (e.g., from the frame of the valve), such that it presses and seals against tissue of the native orifice (e.g., the annulus of the native heart valve) within which it is implanted. To achieve this, upper and lower rims 492 and 494 of the ring of fabric 470 can be attached to valve 500 (e.g., to the frame thereof) with a direct distance between the rims being smaller than the distance between the rims as measured along the fabric. Due to the resilience of weft strand 474, the multiple stretches of the weft strand, which extend between these two attachment points, thus urge fabric 470 to bulge to form pouch 490 as shown. Furthermore, this arrangement can confer this bulging characteristic on each circumferential region of pouch 490 independently, e.g., each stretch of the weft strand behaving, in effect, as a separate strand. That is, should pouch 490 be compressed inward at one place on the circumference of valve 500 (e.g., by an anatomical feature at the implantation site), at other places on the circumference of the valve the pouch may remain bulging. This may advantageously allow pouch 490 to conform to, and therefore seal against, the native anatomy.

For some applications fabric 470 is woven to have a width (i.e., a distance between turns 476 and/or between outermost warp strands 472) appropriate for pouch 490, e.g., as opposed to being woven wider and cut to the appropriate width. Thus, at rims 492 and 494, only turns 476 of weft strand 474 are presented. Moreover, it may be the case that in the entire pouch 490 only two ends of weft strand 474 exist. Because of the stiffer and/or more resilient nature of monofilament weft strand 474, such weaving and orienting of fabric 470 may be advantageous, by reducing or eliminating poking of tissue by that might otherwise be caused were the monofilament be used for warp strands each having free (e.g., cut) ends.

It may be desirable that the interior of pouch 490 fills with blood (e.g., such that the blood coagulates and/or encourages tissue growth therein) in order to further stabilize the anatomy-hugging shape that the pouch initially assumes upon its expansion. Thus, for some applications, fabric 470 has at least some portions which are permeable. Therefore fabric 470 can be manufactured using one or more of the techniques described hereinabove for manufacturing a permeable (e.g., open-weave) fabric. For example, for applications in which fabric 470 is laminated, it can be laminated in a manner that leaves at least part of the fabric clear of lamination, such as being laminated in a pattern, e.g., as described hereinabove with respect to FIG. 18, mutatis mutandis.

There is provided, in accordance with some applications, a method, comprising: (A) arranging lengths of a multifilament yarn (e.g., yarn 472) as warp strands; (B) weaving a fabric (e.g., fabric 470) by passing a monofilament yarn (e.g., yarn 474) between the warp strands in a first direction, and then passing the monofilament yarn between the warp strands in an opposing direction, such that the monofilament yarn forms weft strands connected to each other by turns of the monofilament yarn at lateral edges of the fabric; and (C) forming the fabric into a ring in which the warp strands extend circumferentially around the ring and the weft strands extend along a height of the ring.

Any of the various systems, devices, apparatuses, components, fabrics, yarns, textiles, etc. in this disclosure can be sterilized (e.g., with heat, radiation, ethylene oxide, hydrogen peroxide, etc.) to ensure they are safe for use with patients, and any of the methods herein can include sterilization of the associated system, device, apparatus, component, fabric, yarn, textile, etc. (e.g., with heat, radiation, ethylene oxide, hydrogen peroxide, etc.) as one of the steps of the method.

It should be understood that the use of “and/or” is defined inclusively such that the term “a and/or b” should be read to include the sets: “a and b,” “a or b,” “a,” “b.”

The present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

Although the operations of some of the disclosed examples are described in a particular, sequential order for convenient presentation, it should be understood that this manner of description encompasses rearrangement, unless a particular ordering is required by specific language set forth above. For example, operations or steps described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are discernible by one of ordinary skill in the art.

### Example Applications (Some Non-Limiting Examples of the Concepts Herein are Recited Below)

Example 1. A method, comprising: (A) forming a mixture into a fiber, wherein the mixture includes: (i) a polymer, and (ii) an additive molecule that includes one or more fluorinated end-groups; and (B) incorporating the fiber in a fabric or textile.

Example 2. The method according to example 1, wherein the additive molecule includes (i) a polymeric backbone, and (ii) fluorinated end-groups at at least one end of the backbone

Example 3. The method according to example 2, wherein the polymeric backbone includes polyurethane, and wherein forming the mixture into the fiber comprises forming, into the fiber, the mixture that includes the additive molecule that includes the polymeric backbone that includes polyurethane.

Example 4. The method according to any one of examples 1-3, wherein the polymer is a thermoplastic polyurethane, and forming the mixture into the fiber comprises forming, into the fiber, the mixture that includes the thermoplastic polyurethane.

Example 5. The method according to any one of examples 1-3, wherein the polymer is nylon, and forming the mixture into the fiber comprises forming, into the fiber, the mixture that includes nylon.

Example 6. The method according to any one of examples 1-3, wherein the polymer is an elastomer, and forming the mixture into the fiber comprises forming, into the fiber, the mixture that includes the elastomer.

Example 7. The method according to any one of examples 1-6, wherein forming the mixture into the fiber comprises extruding the mixture into the fiber.

Example 8. The method according to any one of examples 1-6, wherein forming the mixture into the fiber comprises electrospinning the mixture into the fiber.

Example 9. The method according to any one of examples 1-8, wherein incorporating the fiber in the fabric or textile comprises incorporating the fiber in a nonwoven fabric or textile.

Example 10. The method according to any one of examples 1-8, wherein incorporating the fiber in the fabric or textile comprises incorporating the fiber in a blended fabric or textile.

Example 11. The method according to any one of examples 1-10, wherein the mixture includes the additive molecule at 1-5 percent by mass, and forming the mixture into the fiber comprises forming, into the fiber, the mixture that includes the additive molecule at 1-5 percent by mass.

Example 12. The method according to example 11, wherein the mixture includes the additive molecule at 1-4 percent by mass, and forming the mixture into the fiber comprises forming, into the fiber, the mixture that includes the additive molecule at 1-4 percent by mass.

Example 13. The method according to example 12, wherein the mixture includes the additive molecule at 1-3 percent by mass, and forming the mixture into the fiber comprises forming, into the fiber, the mixture that includes the additive molecule at 1-3 percent by mass.

Example 14. The method according to example 13, wherein the mixture includes the additive molecule at 1-2 percent by mass, and forming the mixture into the fiber comprises forming, into the fiber, the mixture that includes the additive molecule at 1-2 percent by mass.

Example 15. The method according to example 13, wherein the mixture includes the additive molecule at 2-3 percent by mass, and forming the mixture into the fiber comprises forming, into the fiber, the mixture that includes the additive molecule at 2-3 percent by mass.

Example 16. The method according to example 11, wherein the mixture includes the additive molecule at 2-5 percent by mass, and forming the mixture into the fiber comprises forming, into the fiber, the mixture that includes the additive molecule at 2-5 percent by mass.

Example 17. The method according to example 16, wherein the mixture includes the additive molecule at 2-4 percent by mass, and forming the mixture into the fiber comprises forming, into the fiber, the mixture that includes the additive molecule at 2-4 percent by mass.

Example 18. The method according to example 17, wherein the mixture includes the additive molecule at 3-4 percent by mass, and forming the mixture into the fiber comprises forming, into the fiber, the mixture that includes the additive molecule at 3-4 percent by mass.

Example 19. The method according to example 16, wherein the mixture includes the additive molecule at 3-5 percent by mass, and forming the mixture into the fiber comprises forming, into the fiber, the mixture that includes the additive molecule at 3-5 percent by mass.

Example 20. The method according to example 19, wherein the mixture includes the additive molecule at 4-5 percent by mass, and forming the mixture into the fiber comprises forming, into the fiber, the mixture that includes the additive molecule at 4-5 percent by mass.

Example 21. The method according to any one of examples 1-20, further comprising incorporating the fabric or textile into a medical implant.

Example 22. The method according to example 21, wherein incorporating the fabric or textile into the medical implant comprises stitching the fabric or textile to a frame of the medical implant.

Example 23. The method according to example 21, wherein incorporating the fabric or textile into the medical implant comprises forming the fabric or textile into a sleeve of the implant.

Example 24. The method according to any one of examples 1-6, wherein the polymer is a polyester, and forming the mixture into the fiber comprises forming, into the fiber, the mixture that includes the polyester.

Example 25. The method according to example 24, wherein the polyester is polyethylene terephthalate, and forming the mixture into the fiber comprises forming, into the fiber, the mixture that includes polyethylene terephthalate.

Example 26. The method according to example 25, wherein the polyethylene terephthalate is a polyethylene terephthalate homopolymer, and forming the mixture into the fiber comprises forming, into the fiber, the mixture that includes polyethylene terephthalate homopolymer.

Example 27. The method according to example 25, wherein the polyethylene terephthalate is a polyethylene terephthalate copolymer, and forming the mixture into the fiber comprises forming, into the fiber, the mixture that includes polyethylene terephthalate copolymer.

Example 28. The method according to any one of examples 1-27, wherein incorporating the fiber into the fabric or textile comprises forming a yarn that includes the fiber, and wherein the method includes incorporating the yarn in the fabric or textile or in a medical implant or medical device.

Example 29. The method according to example 28, wherein the yarn consists substantially of only the fiber, and forming the yarn comprises forming the yarn from substantially only the fiber.

Example 30. The method according to example 28, wherein the fiber is a first fiber, the yarn is a blend of the first fiber and a second fiber, and forming the yarn comprises forming the yarn from the blend of the first fiber and the second fiber.

Example 31. The method according to example 28, wherein forming the yarn comprises spinning the yarn.

Example 32. The method according to example 31, further comprising cutting the fiber into staple fiber, wherein spinning the yarn comprises spinning the staple fiber into the yarn.

Example 33. The method according to example 28, wherein the yarn is a filament yarn, and wherein forming the yarn comprises forming the filament yarn.

Example 34. The method according to example 33, further comprising air texturizing the filament yarn.

Example 35. The method according to example 28, wherein the yarn is a core-spun yarn, and wherein forming the yarn comprises forming the core-spun yarn.

Example 36. The method according to example 28, wherein incorporating the yarn in the fabric or textile comprises producing the fabric or textile by interlacing lengths of the yarn.

Example 37. The method according to example 36, wherein the fabric or textile consists substantially of only the yarn, and producing the fabric or textile comprises producing the fabric or textile by interlacing lengths of substantially only the yarn.

Example 38. The method according to example 36, wherein the yarn is a first yarn, the fabric or textile includes a mixture of the first yarn and a second yarn, and producing the fabric or textile comprises producing the fabric or textile by interlacing lengths of the first yarn and lengths of the second yarn.

Example 39. The method according to example 36, wherein producing the fabric or textile by interlacing lengths of the yarn comprises producing the fabric or textile by weaving lengths of the yarn.

Example 40. The method according to example 36, wherein producing the fabric or textile by interlacing lengths of the yarn comprises producing the fabric or textile by knitting lengths of the yarn.

Example 41. A method, comprising: (A) forming into fiber a mixture that includes: (i) a polymer, and (ii) an additive molecule that includes one or more fluorinated end-groups; and (B) forming a yarn that includes the fiber.

Example 42. The method according to example 41, wherein the additive molecule includes (i) a polymeric backbone, and (ii) fluorinated end-groups at at least one end of the backbone

Example 43. The method according to example 42, wherein the polymeric backbone includes polyurethane, and wherein forming the mixture into the fiber comprises forming, into the fiber, the mixture that includes the additive molecule that includes the polymeric backbone that includes polyurethane.

Example 44. The method according to any one of examples 41-43, wherein the yarn is surgical suture that includes the fiber, and wherein forming the yarn comprises forming the surgical suture that includes the fiber.

Example 45. The method according to any one of examples 41-44, wherein the yarn consists substantially of only the fiber, and forming the yarn comprises forming the yarn from substantially only the fiber.

Example 46. The method according to any one of examples 41-44, wherein the fiber is a first fiber, the yarn includes a mixture of the first fiber and a second fiber, and forming the yarn comprises forming the yarn from the mixture of the first fiber and the second fiber.

Example 47. The method according to any one of examples 41-46, wherein the yarn is a core-spun yarn, and wherein forming the yarn comprises forming the core-spun yarn.

Example 48. The method according to any one of examples 41-47, wherein forming the yarn comprises spinning the yarn.

Example 49. The method according to example 48, further comprising cutting the fiber into staple fiber, wherein spinning the yarn comprises spinning the staple fiber into the yarn.

Example 50. The method according to any one of examples 41-46, wherein the yarn is a filament yarn, and wherein forming the yarn comprises forming the filament yarn.

Example 51. The method according to example 50, further comprising air texturizing the filament yarn.

Example 52. A method, comprising: (A) forming a yarn that includes fiber formed from a mixture of: (i) a polymer, and (ii) an additive molecule that includes one or more fluorinated end-groups; and (B) incorporating the yarn in a fabric or textile.

Example 53. The method according to example 52, wherein the additive molecule that includes (i) a polymeric backbone, and (ii) fluorinated end-groups at at least one end of the backbone.

Example 54. The method according to example 53, wherein the polymeric backbone includes polyurethane, and wherein forming the yarn that includes the fiber comprises forming the yarn that includes the fiber formed from the mixture of the polymer and the additive molecule that includes the polymeric backbone that includes polyurethane.

Example 55. The method according to any one of examples 52-54, wherein the yarn consists substantially of only the fiber, and forming the yarn comprises forming the yarn from substantially only the fiber.

Example 56. The method according to any one of examples 52-54, wherein the fiber is a first fiber, the yarn includes a mixture of the first fiber and a second fiber, and forming the yarn comprises forming the yarn from the mixture of the first fiber and the second fiber.

Example 57. The method according to any one of examples 52-56, wherein the yarn is a core-spun yarn, and wherein forming the yarn comprises forming the core-spun yarn.

Example 58. The method according to any one of examples 52-57, wherein forming the yarn comprises spinning the yarn.

Example 59. The method according to example 58, further comprising cutting the fiber into staple fiber, wherein spinning the yarn comprises spinning the staple fiber into the yarn.

Example 60. The method according to any one of examples 52-56, wherein the yarn is a filament yarn, and wherein forming the yarn comprises forming the filament yarn.

Example 61. The method according to example 60, further comprising air texturizing the filament yarn.

Example 62. The method according to any one of examples 52-61, wherein incorporating the yarn in the fabric or textile comprises producing the fabric or textile by interlacing lengths of the yarn.

Example 63. The method according to example 62, wherein the fabric or textile consists substantially of only the yarn, and producing the fabric or textile comprises producing the fabric or textile by interlacing lengths of substantially only the yarn.

Example 64. The method according to example 62, wherein the yarn is a first yarn, the fabric or textile includes a mixture of the first yarn and a second yarn, and producing the fabric or textile comprises producing the fabric or textile by interlacing lengths of the first yarn and lengths of the second yarn.

Example 65. The method according to example 62, wherein producing the fabric or textile by interlacing lengths of the yarn comprises producing the fabric or textile by at least one of weaving and knitting lengths of the yarn.

Example 66. A method, comprising: (A) obtaining a frame for a medical implant; and (B) dressing the frame with fibers that have been formed from a mixture, the mixture including: (i) a polymer, and (ii) an additive molecule that includes one or more fluorinated end-groups.

Example 67. The method according to example 66, wherein the additive molecule that includes (i) a polymeric backbone, and (ii) fluorinated end-groups at at least one end of the backbone

Example 68. The method according to example 67, wherein the polymeric backbone includes polyurethane, and wherein dressing the frame with the fibers comprises dressing the frame with the fibers that have been formed from the mixture that includes the additive molecule that includes the backbone that includes polyurethane.

Example 69. The method according to any one of examples 66-68, wherein dressing the frame with the fibers comprises at least one of electrospinning the fibers onto the frame.

Example 70. The method according to any one of examples 66-68, wherein dressing the frame with the fibers comprises dressing the frame by stitching a fabric or textile to the frame, wherein the fabric or textile includes the fibers.

Example 71. The method according to any one of examples 66-70, wherein dressing the frame with the fibers comprises dressing only a part of the frame with the fibers.

Example 72. The method according to example 71, wherein the part of the frame is a first part of the frame, and wherein the method further comprises dressing a second part of the frame, different from the first part of the frame, with other fibers that have not been formed from the mixture.

Example 73. An apparatus, comprising a medical implant that comprises fibers that have been formed from a mixture, the mixture comprising: (A) a polymer; and (B) an additive molecule that includes one or more fluorinated end-groups at at least one end of the backbone.

Example 74. The apparatus according to example 73, wherein the additive molecule that includes (i) a polymeric backbone, and (ii) fluorinated end-groups at at least one end of the backbone

Example 75. The apparatus according to example 74, wherein the polymeric backbone comprises polyurethane.

Example 76. The apparatus according to any one of examples 73-75, wherein the implant comprises at least one of a fabric, a textile, a thread, and a suture within which the fibers are included.

Example 77. An apparatus, comprising a fabric or textile that comprises fibers that have been formed from a mixture, the mixture comprising: (A) a polymer; and (B) an additive molecule that includes (i) a polymeric backbone, and (ii) fluorinated end-groups at at least one end of the backbone.

Example 78. The apparatus according to example 77, wherein the polymeric backbone comprises polyurethane.

Example 79. An apparatus, comprising surgical suture that comprises fibers formed into a yarn, the fibers having been formed from a mixture comprising: (A) a polymer; and (B) an additive molecule that includes (i) a polymeric backbone, and (ii) fluorinated end-groups at at least one end of the backbone.

Example 80. The apparatus according to example 79, wherein the polymeric backbone includes polyurethane.

Example 81. A method, comprising: (A) applying, to a fabric or textile, a solution that includes: (i) a solvent, (ii) a polymer, dissolved in the solvent, and (iii)_an additive molecule, dissolved in the solvent, the additive molecule including one or more fluorinated end-groups; and (B) allowing the solvent to evaporate.

Example 82. The method according to example 81, wherein the additive molecule includes (i) a polymeric backbone, and (ii) fluorinated end-groups at at least one end of the backbone.

Example 83. The method according to example 82, wherein the polymeric backbone includes polyurethane, and applying the solution to the fabric or textile comprises applying, to the fabric or textile, the solution that includes the polymeric backbone that includes polyurethane.

Example 84. The method according to any one of examples 81-83, wherein applying the solution to the fabric or textile comprises at least one of dipping the fabric or textile into the solution and spraying the solution onto the fabric or textile.

Example 85. The method according to any one of examples 81-84, wherein the fabric or textile includes natural fibers, and applying the solution to the fabric or textile comprises applying the solution to the fabric or textile that includes the natural fibers.

Example 86. The method according to any one of examples 81-85, wherein the fabric or textile is a blended fabric or textile, and applying the solution to the fabric or textile comprises applying the solution to the blended fabric or textile.

Example 87. The method according to any one of examples 81-86, further comprising preparing the solution by dissolving the polymer and the additive in the solvent.

Example 88. The method according to any one of examples 81-87, wherein the fabric or textile includes synthetic fibers, and applying the solution to the fabric or textile comprises applying the solution to the fabric or textile that includes the synthetic fibers.

Example 89. The method according to example 88, wherein the synthetic fibers comprise polyethylene terephthalate, and applying the solution to the fabric or textile comprises applying the solution to the fabric or textile that includes the synthetic fibers that comprise polyethylene terephthalate.

Example 90. The method according to example 88, wherein the synthetic fibers are formed from the polymer, and applying the solution to the fabric or textile comprises applying the solution to the fabric or textile that includes the synthetic fibers formed from the polymer.

Example 91. The method according to example 88, wherein the polymer is a first polymer, and the synthetic fibers are formed from a second polymer that is different to the first polymer, and applying the solution to the fabric or textile comprises applying the solution to the fabric or textile that includes the synthetic fibers that are formed from the second polymer.

Example 92. The method according to example 91, wherein the second polymer has a different degree of polymerization (DP) to the first polymer.

Example 93. The method according to example 92, wherein the second polymer has a higher DP than the first polymer.

Example 94. The method according to example 92, wherein the second polymer has a lower DP than the first polymer.

Example 95. The method according to example 92, wherein the second polymer is monomerically identical to the first polymer.

Example 96. The method according to example 91, wherein the second polymer is less soluble in the solvent than is the first polymer.

Example 97. The method according to any one of examples 81-96, further comprising incorporating the fabric or textile into a medical implant.

Example 98. The method according to example 97, wherein incorporating the fabric or textile into the medical implant comprises dressing a frame of the implant with the fabric or textile.

Example 99. The method according to example 98, wherein dressing the frame with the fabric or textile comprises stitching the fabric or textile to the frame.

Example 100. The method according to example 98, wherein dressing the frame with the fabric or textile comprises dressing only a part of the frame with the fabric or textile.

Example 101. The method according to example 100, wherein the part of the frame is a first part of the frame, the fabric or textile is a first fabric or textile, and the method further comprises dressing a second part of the frame, different from the first part of the frame, with a second fabric or textile to which the solution has not been applied.

Example 102. The method according to example 97, wherein incorporating the fabric or textile into the medical implant comprises incorporating the fabric or textile into the medical implant subsequently to applying the solution to the fabric or textile.

Example 103. The method according to example 97, wherein incorporating the fabric or textile into the medical implant comprises incorporating the fabric or textile into the medical implant prior to applying the solution to the fabric or textile.

Example 104. A method, comprising: (A) applying, to a fiber, a solution that includes: (a) a solvent, (b) a polymer, dissolved in the solvent, and (c) an additive molecule, dissolved in the solvent, the additive molecule including (i) a polymeric backbone, and (ii) fluorinated end-groups at at least one end of the backbone; (B) forming a residue on the fiber by allowing the solvent to evaporate, the residue including the polymer and the additive molecule; (C) concentrating the additive molecule toward a surface of the residue by heating the residue; and (D) subsequently, allowing the residue to cool.

Example 105. The method according to example 104, wherein heating the residue comprises heating the residue by at least 50 degrees C.

Example 106. The method according to example 104, wherein heating the residue comprises heating the residue to a temperature that is greater than a melting temperature of the polymer and lower than a melting temperature of the fiber.

Example 107. The method according to example 104, wherein the polymeric backbone includes polyurethane, and applying the solution to the fiber comprises applying, to the fiber, the solution that includes the polymeric backbone that includes polyurethane.

Example 108. The method according to example 104, further comprising incorporating the fiber in a yarn, fabric, textile, implant, and/or medical device.

Example 109. The method according to example 108, wherein incorporating the fiber in the yarn, fabric, textile, implant, and/or medical device comprises incorporating the fiber in the yarn, fabric, textile, implant, and/or medical device subsequently to concentrating the additive molecule toward the surface of the residue by heating the residue.

Example 110. The method according to example 108, wherein incorporating the fiber in the yarn, fabric, textile, implant, and/or medical device comprises incorporating the fiber in the yarn, fabric, textile, implant, and/or medical device prior to concentrating the additive molecule toward the surface of the residue by heating the residue.

Example 111. The method according to example 110, wherein applying the solution to the fiber comprises applying the solution to the fiber prior to incorporating the fiber in the yarn, fabric, textile, implant, and/or medical device.

Example 112. The method according to example 110, wherein applying the solution to the fiber comprises applying the solution to the fiber subsequently to incorporating the fiber in the yarn, fabric, textile, implant, and/or medical device.

Example 113. The method according to example 104, wherein heating the residue comprises heating the residue for no more than 1 minute.

Example 114. The method according to example 113, wherein heating the residue comprises heating the residue for no more than 30 seconds.

Example 115. The method according to example 114, wherein heating the residue comprises heating the residue for no more than 10 seconds.

Example 116. The method according to example 115, wherein heating the residue comprises heating the residue for no more than 5 seconds.

Example 117. The method according to example 116, wherein heating the residue comprises heating the residue for no more than 1 second.

Example 118. The method according to example 104, wherein the fiber is a synthetic fiber, and applying the solution to the fiber comprises applying the solution to the synthetic fiber.

Example 119. The method according to example 118, wherein the synthetic fiber comprises polyethylene terephthalate, and applying the solution to the synthetic fiber comprises applying the solution to the synthetic fiber that comprises polyethylene terephthalate.

Example 120. The method according to example 118, wherein the synthetic fiber is formed from the polymer, and applying the solution to the synthetic fiber comprises applying the solution to the synthetic fiber formed from the polymer.

Example 121. The method according to example 118, wherein the polymer is a first polymer, the synthetic fiber is formed from a second polymer that is different from the first polymer, and applying the solution to the synthetic fiber comprises applying the solution to the synthetic fiber formed from the second polymer.

Example 122. The method according to example 121, wherein the second polymer has a different degree of polymerization (DP) than that of the first polymer.

Example 123. The method according to example 122, wherein the second polymer has a higher DP than that of the first polymer.

Example 124. The method according to example 122, wherein the second polymer has a lower DP than that of the first polymer.

Example 125. The method according to example 122, wherein the second polymer is monomerically identical to the first polymer.

Example 126. The method according to example 121, wherein the second polymer is less soluble in the solvent than is the first polymer.

Example 127. The method according to example 121, wherein the second polymer has a different melting temperature than that of the first polymer.

Example 128. The method according to example 127, wherein the second polymer has a higher melting temperature than that of the first polymer.

Example 129. The method according to example 127, wherein the second polymer has a lower melting temperature than that of the first polymer.

Example 130. The method according to example 121, wherein the second polymer has a different glass-transition temperature than that of the first polymer.

Example 131. The method according to example 130, wherein the second polymer has a higher glass-transition temperature than that of the first polymer.

Example 132. The method according to example 130, wherein the second polymer has a lower glass-transition temperature than that of the first polymer.

Example 133. A method, comprising: (A) applying, to a fiber, a solution that includes: (a) a solvent, (b) a polymer, dissolved in the solvent, and (c) an additive molecule, dissolved in the solvent, the additive molecule including (i) a polymeric backbone, and (ii) fluorinated end-groups at at least one end of the backbone; (B) allowing the solvent to evaporate; and (C) forming a yarn that includes the fiber.

Example 134. The method according to example 133, wherein the polymeric backbone includes polyurethane, and wherein applying the solution to the fiber comprises applying, to the fiber, the solution that includes the additive molecule that includes the backbone that includes polyurethane.

Example 135. The method according to example 133, further comprising incorporating the yarn into a fabric.

Example 136. The method according to example 133, wherein the yarn is a medical suture, and wherein forming the yarn comprises forming the medical suture.

Example 137. A method, comprising: (A) applying, to a yarn, a solution that includes: (a) a solvent, (b) a polymer, dissolved in the solvent, and (c) an additive molecule, dissolved in the solvent, the additive molecule including (i) a polymeric backbone, and (ii) fluorinated end-groups at at least one end of the backbone; and (B) allowing the solvent to evaporate.

Example 138. The method according to example 137, wherein the polymeric backbone includes polyurethane, and wherein applying the solution to the yarn comprises applying, to the yarn, the solution that includes the additive molecule that includes the backbone that includes polyurethane.

Example 139. The method according to example 137, further comprising incorporating the yarn into a fabric or textile.

Example 140. The method according to example 137, wherein the yarn is a medical suture, and wherein applying the solution to the yarn comprises applying the solution to the medical suture.

Example 141. A method, comprising: (A) applying, to a fiber, a solution that includes: (i) a solvent, (ii) a polymer, dissolved in the solvent, and (iii) an additive molecule, dissolved in the solvent, the additive molecule including one or more fluorinated end-groups at at least one end of the backbone; (B) allowing the solvent to evaporate; and (C) forming a fabric or textile that includes the fiber.

Example 142. The method according to example 141, wherein the additive molecule, dissolved in the solvent, the additive molecule including (i) a polymeric backbone, and (ii) fluorinated end-groups at at least one end of the backbone

Example 143. The method according to example 142, wherein the polymeric backbone includes polyurethane, and wherein applying the solution to the fiber comprises applying, to the fiber, the solution that includes the additive molecule that includes the backbone that includes polyurethane.

Example 144. The method according to any one of examples 141-143, wherein: (i) allowing the solvent to evaporate comprises forming a residue on the fiber by allowing the solvent to evaporate, the residue including the polymer and the additive molecule, and (ii) the method further comprises: (A) concentrating the additive molecule toward a surface of the residue by heating the residue; and (B) subsequently, allowing the residue to cool.

Example 145. The method according to example 144, wherein forming the fabric or textile comprises forming the fabric or textile subsequently to concentrating the additive molecule toward the surface of the residue by heating the residue.

Example 146. An apparatus, comprising a medical implant that comprises fibers to which a solution has been applied, the solution comprising: (A) a solvent; (B) a polymer, dissolved in the solvent; and (C) an additive molecule, dissolved in the solvent, the additive molecule including (i) a polymeric backbone, and (ii) fluorinated end-groups at at least one end of the backbone.

Example 147. The apparatus according to example 146, wherein the medical implant comprises stitches of a thread that comprises the fibers.

Example 148. The apparatus according to example 146, wherein the polymeric backbone includes polyurethane.

Example 149. The apparatus according to example 146, wherein: (i) the fibers are first fibers, (ii) the solution is a first solution, and (iii) the medical implant further comprises second fibers to which a second solution has been applied, the second solution comprising: (a) a solvent; and (b) a fluorinated polyphosphazene.

Example 150. The apparatus according to any one of examples 146-149, wherein the medical implant comprises a fabric or textile that comprises the fibers.

Example 151. The apparatus according to example 150, wherein: (i) the fibers are first fibers, (ii) the solution is a first solution, (iii) the fabric or textile is a first fabric, and (iv) the medical implant further comprises a second fabric that comprises second fibers to which a second solution has been applied, the second solution comprising: (a) a solvent; and (b) a fluorinated polyphosphazene.

Example 152. The apparatus according to example 150, wherein: (i) the fibers are first fibers, (ii) the solution is a first solution, and (iii) the fabric or textile further comprises second fibers to which a second solution has been applied, the second solution comprising: (a) a solvent; and (b) a fluorinated polyphosphazene.

Example 153. The apparatus according to any one of examples 146-152, wherein the apparatus is for use at a valve of a heart of a subject, the valve having a first leaflet and an opposing leaflet, the heart having a chamber upstream of the valve, and wherein: (i) the implant comprises: (a) an interface; (b) a flexible coaptation portion, coupled to the interface, and comprising: (1) a flexible wire shaped as a loop that defines an aperture therethrough; and (2) a fabric or textile, coupled to the wire and covering at least part of the aperture, the fabric or textile comprising the fibers; and (3) an anchor, and (ii) the apparatus further comprises a delivery system comprising: (A) a shaft, configured to, via engagement with the interface, position the implant in a position in which the interface is at a site in the heart, the coaptation portion extends over the first leaflet toward the opposing leaflet, and the contact face faces the first leaflet; and (B) an anchor driver, configured to secure the implant in the position by anchoring the interface to tissue of the heart by driving the anchor into the tissue.

Example 154. The apparatus according to example 153, wherein the wire is arranged to define the loop as teardrop-shaped.

Example 155. The apparatus according to any one of examples 153-154, wherein the coaptation portion has an open part at which the aperture is not covered by the fabric or textile.

Example 156. The apparatus according to example 155, wherein the coaptation portion has a root that is coupled to the interface, and a tip at an opposite end of the coaptation portion from the root, and wherein the fabric or textile is disposed between the open part and the tip.

Example 157. The apparatus according to any one of examples 146-152, wherein the implant comprises an annuloplasty structure.

Example 158. The apparatus according to example 157, wherein the annuloplasty structure comprises a fabric or textile sleeve that comprises the fibers.

Example 159. The apparatus according to any one of examples 146-152, wherein the implant comprises a prosthetic heart valve.

Example 160. The apparatus according to example 159, wherein the prosthetic heart valve comprises a frame, and a fabric or textile sheet that comprises the fibers, the fabric or textile sheet disposed over at least part of the frame.

Example 161. The apparatus according to example 159, wherein the prosthetic heart valve comprises a thread that stitches components of the prosthetic heart valve together, and wherein the thread comprises the fibers.

Example 162. The apparatus according to example 161, wherein one of the components is a frame, and the thread stitches another of the components to the frame.

Example 163. The apparatus according to example 162, wherein the other of the components is a fabric or textile sheet.

Example 164. The apparatus according to example 162, wherein the other of the components is a prosthetic leaflet.

Example 165. An apparatus, comprising a fabric or textile to which a solution has been applied, the solution comprising: (A) a solvent; (B) a polymer, dissolved in the solvent; and (C) an additive molecule, dissolved in the solvent, the additive molecule including (i) a polymeric backbone, and (ii) fluorinated end-groups at at least one end of the backbone.

Example 166. The apparatus according to example 165, wherein the polymeric backbone includes polyurethane.

Example 167. An apparatus, comprising a surgical suture that comprises fibers formed into a yarn, the fibers having had a solution applied thereto, the solution comprising: (A) a solvent; (B) a polymer, dissolved in the solvent; and (C) an additive molecule, dissolved in the solvent, the additive molecule including (i) a polymeric backbone, and (ii) fluorinated end-groups at at least one end of the backbone.

Example 168. The apparatus according to example 167, wherein the polymeric backbone includes polyurethane.

Example 169. An apparatus, comprising a prosthetic heart valve that comprises: (A) a frame; and (B) prosthetic leaflets, supported by the frame, the prosthetic leaflets having had a solution applied thereto, the solution comprising: (a) a solvent; (b) a polymer, dissolved in the solvent; and (c) an additive molecule, dissolved in the solvent, the additive molecule including (i) a polymeric backbone, and (ii) fluorinated end-groups at at least one end of the backbone.

Example 170. The apparatus according to example 169, wherein the polymeric backbone includes polyurethane.

Example 171. A method, comprising: (A) applying, to a fabric or textile, a solution that includes: (i) a solvent, and (ii) a fluorinated polyphosphazene; and (B) allowing the solvent to evaporate.

Example 172. The method according to example 171, wherein applying the solution to the fabric or textile comprises dipping the fabric or textile into the solution.

Example 173. The method according to example 171, wherein applying the solution to the fabric or textile comprises spraying the solution onto the fabric or textile.

Example 174. The method according to any one of examples 171-173, wherein the fabric or textile includes natural fibers, and applying the solution to the fabric or textile comprises applying the solution to the fabric or textile that includes the natural fibers.

Example 175. The method according to any one of examples 171-174, wherein the fabric or textile is a blended fabric or textile, and applying the solution to the fabric or textile comprises applying the solution to the blended fabric or textile.

Example 176. The method according to any one of examples 171-175, further comprising preparing the solution by dissolving the fluorinated polyphosphazene in the solvent.

Example 177. The method according to any one of examples 171-176, wherein the fabric or textile includes synthetic fibers, and applying the solution to the fabric or textile comprises applying the solution to the fabric or textile that includes the synthetic fibers.

Example 178. The method according to example 177, wherein the synthetic fibers are formed from nylon, and applying the solution to the fabric or textile comprises applying the solution to the fabric or textile that includes the synthetic fibers formed from nylon.

Example 179. The method according to example 177, wherein the synthetic fibers are formed from an elastomer, and applying the solution to the fabric or textile comprises applying the solution to the fabric or textile that includes the synthetic fibers formed from the elastomer.

Example 180. The method according to example 177, wherein the synthetic fibers are formed from a polyester, and applying the solution to the fabric or textile comprises applying the solution to the fabric or textile that includes the synthetic fibers formed from the polyester.

Example 181. The method according to example 180, wherein the polyester is polyethylene terephthalate, and applying the solution to the fabric or textile comprises applying the solution to the fabric or textile that includes the synthetic fibers formed from polyethylene terephthalate.

Example 182. The method according to example 177, wherein the synthetic fibers are formed from a thermoplastic polyurethane, and applying the solution to the fabric or textile comprises applying the solution to the fabric or textile that includes the synthetic fibers formed from the thermoplastic polyurethane.

Example 183. The method according to any one of examples 171-182, further comprising incorporating the fabric or textile into a medical implant.

Example 184. The method according to example 183, wherein incorporating the fabric or textile into the medical implant comprises dressing a frame of the implant with the fabric or textile.

Example 185. The method according to example 184, wherein dressing the frame with the fabric or textile comprises stitching the fabric or textile to the frame.

Example 186. The method according to example 184, wherein dressing the frame with the fabric or textile comprises dressing only a part of the frame with the fabric or textile.

Example 187. The method according to example 186, wherein the part of the frame is a first part of the frame, the fabric or textile is a first fabric or textile, and the method further comprises dressing a second part of the frame, different from the first part of the frame, with a second fabric or textile to which the solution has not been applied.

Example 188. The method according to example 183, wherein incorporating the fabric or textile into the medical implant comprises incorporating the fabric or textile into the medical implant subsequently to applying the solution to the fabric or textile.

Example 189. The method according to example 183, wherein incorporating the fabric or textile into the medical implant comprises incorporating the fabric or textile into the medical implant prior to applying the solution to the fabric or textile.

Example 190. A method, comprising: (A) applying, to a fiber, a solution that includes: (i) a solvent, and (ii) a fluorinated polyphosphazene; (B) allowing the solvent to evaporate; and (C) forming a yarn that includes the fiber.

Example 191. The method according to example 190, further comprising incorporating the yarn into a fabric or textile.

Example 192. The method according to example 190, wherein the yarn is a medical suture, and wherein forming the yarn comprises forming the medical suture.

Example 193. A method, comprising: (A) applying, to a yarn, a solution that includes: (i) a solvent, and (ii) a fluorinated polyphosphazene; and (B) allowing the solvent to evaporate.

Example 194. The method according to example 193, further comprising incorporating the yarn into a fabric or textile.

Example 195. The method according to example 193, wherein the yarn is a medical suture, and wherein applying the solution to the yarn comprises applying the solution to the medical suture.

Example 196. An apparatus, comprising a medical implant that comprises fibers to which a solution has been applied, the solution comprising: (i) a solvent; and (ii) a fluorinated polyphosphazene.

Example 197. The apparatus according to example 196, wherein: (A) the fibers are first fibers, (B) the solution is a first solution, and (C) the medical implant further comprises second fibers to which a second solution has been applied, the second solution comprising: (a) a second solvent, (b) a polymer, dissolved in the second solvent, and (c) an additive molecule, dissolved in the second solvent, the additive molecule including (i) a polymeric backbone, and (ii) fluorinated end-groups at at least one end of the backbone.

Example 198. The apparatus according to any one of examples 196-197, wherein the medical implant comprises stitches of a thread that comprises the fibers.

Example 199. The apparatus according to any one of examples 196-197, wherein the medical implant comprises a thread that comprises the fibers.

Example 200. The apparatus according to example 196, wherein the medical implant comprises a fabric or textile that comprises the fibers.

Example 201. The apparatus according to example 200, wherein: (A) the fibers are first fibers, (B) the solution is a first solution, (C) the fabric or textile is a first fabric, and (D) the medical implant further comprises a second fabric that comprises second fibers to which a second solution has been applied, the second solution comprising: (a) a second solvent, (b) a polymer, dissolved in the second solvent, and (c) an additive molecule, dissolved in the second solvent, the additive molecule including (i) a polymeric backbone, and (ii) fluorinated end-groups at at least one end of the backbone.

Example 202. The apparatus according to example 200, wherein: (A) the fibers are first fibers, (B) the solution is a first solution, and (C) the fabric or textile further comprises second fibers to which a second solution has been applied, the second solution comprising: (a) a second solvent, (b) a polymer, dissolved in the second solvent, and (c) an additive molecule, dissolved in the second solvent, the additive molecule including (i) a polymeric backbone, and (ii) fluorinated end-groups at at least one end of the backbone.

Example 203. The apparatus according to any one of examples 196-202, wherein the apparatus is for use at a valve of a heart of a subject, the valve having a first leaflet and an opposing leaflet, the heart having a chamber upstream of the valve, and wherein: (A) the implant comprises: (a) an interface; (b) a flexible coaptation portion, coupled to the interface, and comprising: (i) a flexible wire shaped as a loop that defines an aperture therethrough; and (ii) a fabric or textile, coupled to the wire and covering at least part of the aperture, the fabric or textile comprising the fibers; and (iii) an anchor, and (B) the apparatus further comprises a delivery system comprising: (a) a shaft, configured to, via engagement with the interface, position the implant in a position in which the interface is at a site in the heart, and the coaptation portion extends over the first leaflet toward the opposing leaflet; and (b) an anchor driver, configured to secure the implant in the position by anchoring the interface to tissue of the heart by driving the anchor into the tissue.

Example 204. The apparatus according to example 203, wherein the wire is arranged to define the loop as teardrop-shaped.

Example 205. The apparatus according to example 203, wherein the coaptation portion has an open part at which the aperture is not covered by the fabric or textile.

Example 206. The apparatus according to example 205, wherein the coaptation portion has a root that is coupled to the interface, and a tip at an opposite end of the coaptation portion from the root, and wherein the fabric or textile is disposed between the open part and the tip.

Example 207. The apparatus according to any one of examples 196-202, wherein the implant comprises an annuloplasty structure.

Example 208. The apparatus according to example 207, wherein the annuloplasty structure comprises a fabric sleeve that comprises the fibers.

Example 209. The apparatus according to any one of examples 196-202, wherein the implant comprises a prosthetic heart valve.

Example 210. The apparatus according to example 209, wherein the prosthetic heart valve comprises a frame, and a fabric or textile sheet that comprises the fibers, the fabric or textile sheet disposed over at least part of the frame.

Example 211. The apparatus according to example 209, wherein the prosthetic heart valve comprises a thread that stitches components of the prosthetic heart valve together, and wherein the thread comprises the fibers.

Example 212. The apparatus according to example 211, wherein one of the components is a frame, and the thread stitches another of the components to the frame.

Example 213. The apparatus according to example 212, wherein the other of the components is a fabric or textile sheet.

Example 214. The apparatus according to example 212, wherein the other of the components is a prosthetic leaflet.

Example 215. An apparatus, comprising a surgical suture, comprising fibers formed into a yarn, the fibers having had a solution applied thereto, the solution comprising: (A) a solvent; and (B) a fluorinated polyphosphazene.

Example 216. An apparatus, comprising a prosthetic heart valve that comprises: (A) a frame; and (B) prosthetic leaflets, supported by the frame, the prosthetic leaflets having had a solution applied thereto, the solution comprising: (i) a solvent; and (ii) a fluorinated polyphosphazene.

Example 217. An apparatus, comprising a fabric or textile to which a solution has been applied, the solution comprising: (A) a solvent; and (B) a fluorinated polyphosphazene.

Example 218. A method of forming a stabilized fabric, the method comprising: (A) weaving a fabric from multiple strands of implantable yarn, each strand of implantable yarn at least partially coated with a thermoplastic material; and (B) heating junction points in the fabric to fix intersecting strands of yarn to each other, thereby to reinforce the structure of the fabric.

Example 219. The method according to example 218, further comprising at least partially coating each of the multiple strands of yarn with the thermoplastic material.

Example 220. The method according to example 218 or example 219, wherein multiple strands of implantable yarn include polyethylene.

Example 221. The method according to any one of examples 218 to 220, wherein the thermoplastic material includes thermoplastic polyurethane.

Example 222. The method according to any one of examples 218 to 221, wherein the thermoplastic material includes fluorinated ethylene propylene.

Example 223. The method according to any one of examples 218 to 222, wherein the thermoplastic material includes ultrahigh molecular weight polyethylene.

Example 224. The method according to any one of examples 218 to 223, wherein the thermoplastic material includes expanded polytetrafluoroethylene

Example 225. The method according to any one of examples 219 to 224, wherein the at least partially coating comprises coating the entirety of the exterior of the multiple strands of the implantable yarn with the thermoplastic material.

Example 226. The method according to example 225, wherein the coating comprises: (i) forming an implantable yarn from a plurality of implantable fibers; and (ii) coating the formed implantable yarn with the thermoplastic material.

Example 227. The method according to example 226, wherein the coating of the formed implantable yarn comprises co-extruding the formed implantable yarn and the thermoplastic material.

Example 228. The method according to example 225, wherein the coating comprises: (i) coating a plurality of implantable fibers with the thermoplastic material; and (ii) forming the implantable yarn from the plurality of implantable fibers, following coating thereof.

Example 229. The method according to example 225, wherein the at least partially coating of the plurality of implantable fibers comprises co-extruding the plurality of implantable fibers and the thermoplastic material.

Example 230. The method according to any one of examples 219 to 224, wherein the at least partially coating comprises spinning a strand formed of the thermoplastic material about a core formed of the implantable yarn.

Example 231. The method according to any one of examples 219 to 224, wherein the at least partially coating comprises twisting together at least one strand of the implantable yarn and at least one strand of the thermoplastic material, when they are disposed parallel to one another.

Example 232. The method according to any one of examples 218 to 231, wherein the weaving comprises weaving a leno fabric defining a plurality of windows.

Example 233. The method according to example 232, wherein the greatest dimension of the plurality of windows is not greater than 2 mm.

Example 234. The method according to example 232, wherein the greatest dimension of the plurality of windows is not greater than 1.8 mm.

Example 235. The method according to example 232, wherein the greatest dimension of the plurality of windows is not greater than 1.5 mm.

Example 236. The method according to example 232, wherein the greatest dimension of the plurality of windows is not greater than 1.2 mm.

Example 237. The method according to example 232, wherein the greatest dimension of the plurality of windows is not greater than 1 mm.

Example 238. The method according to any one of examples 218 to 237, wherein, following the heating, the fabric has a thickness not greater than 80 μm.

Example 239. The method according to any one of examples 218 to 237, wherein, following the heating, the fabric has a thickness not greater than 75 μm.

Example 240. The method according to any one of examples 218 to 237, wherein, following the heating, the fabric has a thickness not greater than 70 μm.

Example 241. The method according to any one of examples 218 to 237, wherein, following the heating, the fabric has a thickness not greater than 65 μm

Example 242. The method according to any one of examples 218 to 241, wherein the heating comprises heat pressing the fabric to heat the junction points.

Example 243. The method according to any one of examples 218 to 241, wherein the heating comprises locally heating each of the junction points.

Example 244. The method according to any one of examples 218 to 243, further comprising, following the heating, laminating at least one side of the fabric.

Example 245. The method according to example 244, wherein the laminating comprises laminating only one side of the fabric.

Example 246. The method according to example 244, wherein the laminating comprises laminating both sides of the fabric.

Example 247. The method according to any one of examples 244 to 246, wherein the laminating comprises laminating the fabric in a pattern to form laminated regions and non-laminated regions.

Example 248. A stabilized fabric, comprising a woven structure formed from a plurality of strands of yarn, each including an implantable yarn, the exterior of which is at least partially coated with a thermoplastic material, wherein at least one junction point between some of the plurality of strands of yarn in the woven structure having been heated to retain the relative positioning of the some of the plurality of strands of yarn.

Example 249. The stabilized fabric according to example 248, wherein the implantable yarn includes polyethylene.

Example 250. The stabilized fabric according to example 248 or of example 249, wherein the thermoplastic material includes thermoplastic polyurethane.

Example 251. The stabilized fabric according to any one of examples 248 to 250, wherein the thermoplastic material includes fluorinated ethylene propylene.

Example 252. The stabilized fabric according to any one of examples 248 to 251, wherein the thermoplastic material includes ultrahigh molecular weight polyethylene.

Example 253. The stabilized fabric according to any one of examples 248 to 252, wherein the thermoplastic material includes expanded polytetrafluoroethylene

Example 254. The stabilized fabric according to any one of examples 248 to 253, wherein the exterior of the implantable yarn is entirely coated with the thermoplastic material.

Example 255. The stabilized fabric according to example 254, wherein the implantable yarn is formed of a plurality of implantable fibers, the exterior of each of the implantable fibers being fully coated with the thermoplastic material.

Example 256. The stabilized fabric according to any one of examples 248 to 253, wherein the yarn comprises a core of the implantable yarn, about which is spun a strand of the thermoplastic material.

Example 257. The stabilized fabric according to any one of examples 248 to 253, wherein the yarn comprises a strand of the implantable yarn twisted together with a strand of the thermoplastic material.

Example 258. The stabilized fabric according to any one of examples 248 to 257, wherein the woven structure comprises a leno fabric structure defining a plurality of windows.

Example 259. The stabilized fabric according to example 258, wherein the greatest dimension of the plurality of windows is not greater than 2 mm.

Example 260. The stabilized fabric according to example 258, wherein the greatest dimension of the plurality of windows is not greater than 1.8 mm.

Example 261. The stabilized fabric according to example 258, wherein the greatest dimension of the plurality of windows is not greater than 1.5 mm.

Example 262. The stabilized fabric according to example 258, wherein the greatest dimension of the plurality of windows is not greater than 1.2 mm.

Example 263. The stabilized fabric according to example 258, wherein the greatest dimension of the plurality of windows is not greater than 1 mm.

Example 264. The stabilized fabric according to any one of examples 248 to 263, wherein the fabric has a thickness not greater than 80 μm.

Example 265. The stabilized fabric according to any one of examples 248 to 263, wherein the fabric has a thickness not greater than 75 μm.

Example 266. The stabilized fabric according to any one of examples 248 to 263, wherein the fabric has a thickness not greater than 70 μm.

Example 267. The stabilized fabric according to any one of examples 248 to 263, wherein the fabric has a thickness not greater than 65 μm

Example 268. The stabilized fabric according to any one of examples 248 to 267, wherein the fabric is laminated on at least one side thereof.

Example 269. The stabilized fabric according to example 268, wherein the fabric is laminated only on one side thereof.

Example 270. The stabilized fabric according to example 268, wherein the fabric is laminated on both sides thereof.

Example 271. The stabilized fabric according to any one of examples 268 to 270, wherein the fabric is laminated in a pattern which forms laminated regions and non-laminated regions.

Example 272. A method of forming a stabilized fabric, the method comprising: (A) forming a mesh fabric from strands of an implantable yarn; and (B) stabilizing the mesh fabric by coating at least one side of the mesh fabric with a thermoplastic polymer.

Example 273. The method according to example 272, wherein the forming of the mesh fabric comprises forming the mesh fabric from an implantable yarn including polyethylene.

Example 274. The method according to example 272 or example 273, wherein the forming of the mesh fabric comprises weaving a leno fabric defining a plurality of windows as the mesh fabric.

Example 275. The method according to example 274, wherein the greatest dimension of the plurality of windows is not greater than 2 mm.

Example 276. The method according to example 274, wherein the greatest dimension of the plurality of windows is not greater than 1.8 mm.

Example 277. The method according to example 274, wherein the greatest dimension of the plurality of windows is not greater than 1.5 mm.

Example 278. The method according to example 274, wherein the greatest dimension of the plurality of windows is not greater than 1.2 mm.

Example 279. The method according to example 274, wherein the greatest dimension of the plurality of windows is not greater than 1 mm.

Example 280. The method according to example 272 or example 273, wherein the forming of the mesh fabric comprises knitting a mesh knit as the mesh fabric.

Example 281. The method according to any one of examples 272 to 280, wherein the coating comprises coating the at least one side of the mesh fabric with the thermoplastic polymer.

Example 282. The method according to any one of examples 272 to 281, wherein the thermoplastic polymer includes thermoplastic polyurethane.

Example 283. The method according to any one of examples 272 to 282, wherein the thermoplastic polymer includes fluorinated ethylene propylene.

Example 284. The method according to any one of examples 272 to 283, wherein the thermoplastic polymer includes ultrahigh molecular weight polyethylene.

Example 285. The method according to any one of examples 272 to 284, wherein the thermoplastic polymer includes expanded polytetrafluoroethylene.

Example 286. The method according to any one of examples 272 to 285, wherein the coating comprises spray coating the mesh fabric with a solution including the thermoplastic polymer.

Example 287. The method according to any one of examples 272 to 285, wherein the coating comprises dip coating the mesh fabric with a solution including the thermoplastic polymer.

Example 288. The method according to example 286 or example 287, wherein the coating comprises forming the solution including the thermoplastic polymer and a solvent.

Example 289. The method according to example 288, wherein the solvent includes tetrahydrofuran.

Example 290. The method according to example 288 or example 289, wherein the solvent includes dimethylacetamide.

Example 291. The method according to any one of examples 288 to 290, wherein the solvent includes acetone.

Example 292. The method according to any one of examples 288 to 291, wherein the coating comprises, following application of the solution onto the mesh fabric: (i) allowing the solvent to evaporate; (ii) washing and drying the mesh fabric; and (iii) heat pressing the mesh fabric.

Example 293. The method according to any one of examples 272 to 285, wherein the coating comprises laminating the mesh fabric.

Example 294. The method according to example 293, wherein the laminating comprises laminating the fabric in a pattern to form laminated regions and non-laminated regions.

Example 295. The method according to any one of examples 272 to 294, wherein the coating comprises coating only one side of the mesh fabric.

Example 296. The method according to any one of examples 272 to 294, wherein the coating comprises coating both sides of the mesh fabric.

Example 297. The method according to any one of examples 272 to 296, wherein, following the coating, the fabric has a thickness not greater than 80 μm.

Example 298. The method according to any one of examples 272 to 296, wherein, following the coating, the fabric has a thickness not greater than 75 μm.

Example 299. The method according to any one of examples 272 to 296, wherein, following the coating, the fabric has a thickness not greater than 70 μm.

Example 300. The method according to any one of examples 272 to 296, wherein, following the coating, the fabric has a thickness not greater than 65 μm

Example 301. The method according to any one of examples 272 to 300, further comprising, following the coating, at least one of scouring and sterilizing the mesh fabric.

Example 302. A stabilized fabric comprising a mesh fabric formed from strands of an implantable yarn, at least one side of the mesh fabric being coated with a thermoplastic polymer.

Example 303. The stabilized fabric according to example 302, wherein the implantable yarn comprises polyethylene.

Example 304. The stabilized fabric according to example 302 or example 303, wherein the mesh fabric comprises a leno fabric defining a plurality of windows.

Example 305. The stabilized fabric according to example 304, wherein the greatest dimension of the plurality of windows is not greater than 2 mm.

Example 306. The stabilized fabric according to example 304, wherein the greatest dimension of the plurality of windows is not greater than 1.8 mm.

Example 307. The stabilized fabric according to example 304, wherein the greatest dimension of the plurality of windows is not greater than 1.5 mm.

Example 308. The stabilized fabric according to example 304, wherein the greatest dimension of the plurality of windows is not greater than 1.2 mm.

Example 309. The stabilized fabric according to example 304, wherein the greatest dimension of the plurality of windows is not greater than 1 mm.

Example 310. The stabilized fabric according to example 302 or example 303, wherein the mesh fabric comprises a mesh knit.

Example 311. The stabilized fabric according to any one of examples 302 to 310, wherein the thermoplastic polymer includes thermoplastic polyurethane.

Example 312. The stabilized fabric according to any one of examples 302 to 311, wherein the thermoplastic polymer includes fluorinated ethylene propylene.

Example 313. The stabilized fabric according to any one of examples 302 to 312, wherein the thermoplastic polymer includes ultrahigh molecular weight polyethylene.

Example 314. The stabilized fabric according to any one of examples 302 to 313, wherein the thermoplastic polymer includes expanded polytetrafluoroethylene.

Example 315. The stabilized fabric according to any one of examples 302 to 314, wherein the thermoplastic polymer forms a laminate over the at least one side of the mesh fabric.

Example 316. The stabilized fabric according to example 315, wherein the laminate coating is patterned and defines laminated regions and non-laminated regions.

Example 317. The stabilized fabric according to any one of examples 302 to 316, wherein the mesh fabric is coated only on one side thereof.

Example 318. The stabilized fabric according to any one of examples 302 to 316, wherein the mesh fabric is coated on both sides thereof.

Example 319. The stabilized fabric according to any one of examples 302 to 318, wherein the fabric has a thickness not greater than 80 μm.

Example 320. The stabilized fabric according to any one of examples 302 to 318, wherein the fabric has a thickness not greater than 75 μm.

Example 321. The stabilized fabric according to any one of examples 302 to 318, wherein the fabric has a thickness not greater than 70 μm.

Example 322. The stabilized fabric according to any one of examples 302 to 318, wherein the fabric has a thickness not greater than 65 μm

Example 323. A method, comprising: (A) arranging lengths of a multifilament yarn as warp strands; (B) weaving a fabric by passing a monofilament yarn between the warp strands in a first direction, and then passing the monofilament yarn between the warp strands in an opposing direction, such that the monofilament yarn forms weft strands connected to each other by turns of the monofilament yarn at lateral edges of the fabric; and (C) forming the fabric into a ring in which the warp strands extend circumferentially around the ring and the weft strands extend along a height of the ring.

Example 324. The method according to example 323, wherein the weaving includes weaving the monofilament yarn together with additional lengths of multifilament yarn as the weft strands.

Example 325. The method according to example 323 or example 324, wherein the weaving includes weaving a nitinol fiber as, or with, the monofilament yarn.

Example 326. The method according to any one of examples 323 to 325, further comprising scouring the fabric.

Example 327. The method according to any one of examples 323 to 326, further comprising heat pressing the fabric.

Example 328. The method according to any one of examples 323 to 327, further comprising sterilizing the fabric.

Example 329. The method according to any one of examples 323 to 328, further comprising, prior to the attaching, at least partially laminating at least one side of the fabric with a thermoplastic polymer.

Example 330. The method according to example 329, wherein the at least partially laminating comprises laminating the fabric in a pattern to form laminated regions and non-laminated regions.

Example 331. The method according to example 330, wherein the laminating in a pattern comprises laminating in a pattern such that the laminated regions are not parallel to the warp strands or the weft strands.

Example 332. The method according to example 330, wherein the laminating in a pattern comprises laminating such that the laminated regions form a trellis structure and the non-laminated regions are rhombus shaped.

Example 333. The method according to any one of examples 330 to 332, wherein the laminating comprises laminating using a thermoplastic polymer.

Example 334. The method according to example 333, wherein the thermoplastic polymer includes thermoplastic polyurethane.

Example 335. The method according to any one of examples 333 to 334, wherein the thermoplastic polymer includes fluorinated ethylene propylene.

Example 336. The method according to any one of examples 333 to 335, wherein the thermoplastic polymer includes ultrahigh molecular weight polyethylene.

Example 337. The method according to any one of examples 333 to 336, wherein the thermoplastic polymer includes expanded polytetrafluoroethylene.

Example 338. The method according to any one of examples 330 to 337, wherein forming the fabric into the ring comprises forming the fabric into the ring by forming each of the lateral edges of the fabric into a respective rim of the rim.

Example 339. A method of forming a medical implant, the method comprising: (A) forming a circumferential length of fabric according to the method of any one of examples 323 to 338; and (B) attaching the circumferential length of fabric to an implantable frame, such that the warp strands of the fabric extend about a circumference of the implantable frame.

Example 340. An apparatus comprising: (A) lengths of multifilament yarn as warp strands; (B) a length of a monofilament yarn stretched between the warp strands in a first direction, and then folded and stretched between the warp strands in an opposing direction to form a fabric, such that the monofilament yarn forms weft strands attached by folds of the monofilament yarn at lateral edges of the fabric, (C) opposing ends of each of the warp strands being attached to one another to form a circumferential structure of the fabric, having the warp strands forming circumferences of the circumferential structure and having the weft strands extending perpendicularly to the circumferences, with the folds of the monofilament yarn disposed at circumferential ends of the circumferential structure.

Example 341. The apparatus according to example 340, wherein the weft strands include the monofilament yarn together with additional lengths of multifilament yarn.

Example 342. The apparatus according to example 340 or example 341, wherein the weft strands include a nitinol fiber as, or with, the monofilament yarn.

Example 343. The apparatus according to any one of examples 340 to 342, wherein the fabric is at least partially laminated on at least one side thereof with a thermoplastic polymer.

Example 344. The apparatus according to example 343, wherein the fabric is laminated in a pattern to form laminated regions and non-laminated regions.

Example 345. The apparatus according to example 344, wherein, in the pattern, the laminated regions are not parallel to the warp strands or the weft strands.

Example 346. The apparatus according to example 344, wherein, in the pattern, the laminated regions form a trellis structure and the non-laminated regions are rhombus shaped.

Example 347. The apparatus according to any one of examples 344 to 346, wherein the thermoplastic polymer includes thermoplastic polyurethane.

Example 348. The apparatus according to any one of examples 344 to 347, wherein the thermoplastic polymer includes fluorinated ethylene propylene.

Example 349. The apparatus according to any one of examples 344 to 348, wherein the thermoplastic polymer includes ultrahigh molecular weight polyethylene.

Example 350. The apparatus according to any one of examples 344 to 349, wherein the thermoplastic polymer includes expanded polytetrafluoroethylene.

Example 351. The apparatus according to any one of examples 344 to 350, wherein the laminated surface of the fabric is disposed on an interior side of the circumferential structure.

Example 352. A medical implant, comprising: (A) an implantable frame; and (B) a circumferential structure according to the apparatus of any one of examples 340 to 351, attached about a circumference of the implantable frame.

1. An apparatus, comprising an implant for implantation at a heart valve of a subject, the implant comprising:
a frame; and
a textile mounted on the frame, the textile comprising fibers formed from a mixture of:
a polymer; and
an additive molecule that comprises:
a polyurethane backbone, and
one or more fluorinated end-groups at at least one end of the backbone.

2. The apparatus according to claim 1, wherein the textile comprises an air texturized yarn that includes the fibers.

3. The apparatus according to claim 1, wherein the textile comprises a core-spun yarn that includes the fibers.

4. The apparatus according to claim 1, wherein the textile comprises a yarn that is formed from (i) the fibers formed from the mixture, and (ii) other fibers that are not formed from the mixture.

5. The apparatus according to claim 1, wherein the mixture included the additive molecule at 1-5 percent by mass.

6. The apparatus according to claim 1, wherein the textile is an electrospun textile.

7. The apparatus according to claim 1, wherein the textile is a woven textile.

8. The apparatus according to claim 1, wherein the textile is a knitted textile.

9. The apparatus according to claim 1, wherein the textile is a nonwoven textile.

10. The apparatus according to claim 1, wherein the textile is a blended textile.

11. The apparatus according to claim 1, wherein the polymer is an elastomer.

12. The apparatus according to claim 1, wherein the additive molecule is configured to promote a thin layer of endothelium to form a smooth benign surface on the textile while preventing overthickening of the layer on the textile.

13. The apparatus according to claim 1, wherein the textile is a fabric with which at least part of the frame is dressed.

14. The apparatus according to claim 1, wherein the textile is a suture that is stitched onto at least part of the frame.

15. The apparatus according to claim 1, wherein the implant is an annuloplasty structure.

16. The apparatus according to claim 1, wherein the implant is a prosthetic valve, the frame is a stent frame that is dressed with the textile.

17. The apparatus according to claim 16, wherein the stent frame is dressed with the textile in a manner that directs blood flow through the prosthetic valve.

18. The apparatus according to claim 16, wherein the stent frame is dressed with the textile in a manner that inhibits paravalvular leakage between the prosthetic valve and the heart valve.

19. The apparatus according to claim 1, wherein:
the heart valve has a first leaflet and an opposing leaflet, a heart chamber being upstream of the heart valve,
the frame is defined at least in part by a flexible wire, and
the implant comprises:
an interface; and
a flexible coaptation portion, coupled to the interface, and comprising:
a loop of the flexible wire, the loop defining an aperture therethrough; and
the textile, coupled to the wire and covering at least part of the aperture; and
an anchor, configured to anchor the interface to a site in the heart such that the coaptation portion extends over the first leaflet toward the opposing leaflet in a manner in which, during ventricular systole of the heart, the first leaflet and the opposing leaflet coapt against the coaptation portion.

20. The apparatus according to claim 19, wherein the coaptation portion has an open part in which the aperture is not covered by the textile.

21. The apparatus according to claim 19, further comprising a delivery system comprising:
a shaft, configured to, via engagement with the interface, transluminally position the interface is at the site, and
an anchor driver, configured to anchor the interface to the site by driving the anchor into the tissue.
